(19) United States
(12) Patent Application Publication
(10) Pub. No.: US 2008/0096859 A1
(43) Pub. Date: Apr. 24, 2008

(54) Compositions and Methods for Treating Ophthalmic Diseases

(76) Inventors: Kathleen A. Sullivan, Springfield, NJ (US); Catherine J. Thut, Glenside, PA (US)

Correspondence Address: MERCK AND CO., INC, P O BOX 2000, RAHWAY, NJ, 07065-0907, US

(21) Appl. No.: 11/662,110

(22) PCT Filed: Sep. 27, 2005
(86) PCT No.: PCT/US05/34506

§ 371 Date: Mar. 6, 2007

Related U.S. Application Data
(60) Provisional application No. 60/619,913, filed on Oct. 1, 2004.

(51) Int. Cl. A61K 31/4196; A61K 31/4178; A61K 31/437; A61K 31/454; A61P 27/02

(52) U.S. Cl. 514/210.01; 514/300; 514/317; 514/383; 514/397

(57) ABSTRACT

This invention relates to CXCR4 inhibitors and their use in treating and/or preventing a variety of angiogenic, microvascular and ocular disorders including primary indications for diabetic retinopathy, macular degeneration (such as wet or neovascular age-related macular degeneration (AMD) and dry or atrophic AMD), macular edema, and secondary indications for inhibiting tumor vascularization, and corneal and iris neovascularization.

### BACKGROUND OF THE INVENTION

The inappropriate growth of blood vessels in the adult contributes to a variety of pathogenic conditions. For instance, tumor growth is supported by the formation of new vessels to provide oxygen and nutrients. In the eye, growth of the normally quiescent vasculature can lead to retinal damage and visual impairment or blindness. Neovascular (or wet) AMD and diabetic retinopathy, are the most prevalent disorders of the ocular vasculature and are the leading causes of blindness in the developed world. The current standard of care for ocular neovascular disease is laser surgery (photocoagulation or photodynamic therapy). Unfortunately, laser surgery is modestly effective and only indicated in a small subset of the AMD patient population. For DR, laser treatment is effective in reducing blood vessel growth in many patients, but the laser treatment destroys portions of the peripheral retina and may itself cause visual loss. Therefore, additional treatment modalities need to be explored for the treatment of ocular neovascular diseases.

One means of treating ocular neovascular disease would be to directly target the vasculature and inhibit new blood vessel formation. Many groups are focused on developing anti-angiogenic therapeutics based on inhibiting VEGF signaling (Federico Cappuzzo et al., Expert Opinion in Emerging Drugs 8, 179-192 (2003); Massimo Cristofanilli et al., Nature Reviews. Drug Discovery 1: 415-426 (2002) and Andreas Bikfalvi and Roy Bicknell, Trends in Pharmacological Sciences 23: 576-582 (2002)). However, a variety of other signaling pathways are also involved in modulating angiogenesis. One such pathway involves the chemokine SDF-1 and its receptor CXCR4. Mice deficient for either CXCR4 or SDF-1 have defects in the formation of the large blood vessels that supply the organs of the GI tract and the brain, see Yong-Rui Zou, et. al., Nature 393, 591-594 (1998); Kazunobu Tachibana et. al., Nature 393, 595-599 (1998) and Takashi Nagasawa et. al., Nature 382, 635-638 (1996). In addition, subcutaneous injection of SDF-1 causes localized neovascularization (Rosalba Salcedo et al., American Journal of Pathology 154: 1125-1135 (1999)).

A role for CXCR4 in ocular neovascular disease is suggested by its expression pattern in the eye. mRNA for CXCR4 has been shown to be expressed in vascular endothelial cells that are a component of blood vessels and capillaries (Ombretta Salvucci et al., Blood 99: 2703-2711 (2002). In addition, CXCR4 is expressed in the retinal pigmented epithelium (RPE) that lies between the choroidal vasculature and the retinal neurons (Isabel Crane et al., Journal of Immunology 165: 4372-4378 (2000). Thus, CXCR4 is in the right location to influence the process of CNV and diabetic retinopathy.

It is also possible that CXCR4 may play a role in the non-neovascular form of AMD, also called dry or atrophic AMD. There is evidence to suggest that inflammation may contribute to the pathogenesis of dry AMD (Philip Penfold et al., Progress in Retinal and Eye Research 20: 385-414 (2001), and CXCR4 has been implicated in the inflammatory process (Nicholas Lukacs et al., American Journal of Pathology 160: 1353-1360 (2002); Patrick Matthys et al., Journal of Immunology 167: 4686-4692 (2001) and Jose-Angel Gonzalo et al., Journal of Immunology 165: 499-508 (2000).

This invention relates to CXCR4 antagonists and their use to inhibit CXCR4 signaling thereby reducing the extent of neovascularization and/or inflammation in a variety of angiogenic, microvascular and ocular diseases and/or preventing said diseases.

### BRIEF DESCRIPTION OF THE FIGURES

FIG. 1: shows that periocular injection of a CXCR4 inhibitor reduces ocular neovascularization in a Mouse Model

FIG. 2: shows that intravitreal injection of a CXCR4 inhibitor reduces ocular neovascularization in a Mouse Model

### SUMMARY OF THE INVENTION

This invention relates to CXCR4 inhibitors and their use in treating and/or preventing a variety of angiogenic, microvascular and ocular disorders including primary indications for diabetic retinopathy, macular degeneration (such as wet or neovascular age-related macular degeneration (AMD) and dry or atrophic AMD), macular edema, and secondary indications for inhibiting tumor vascularization, and corneal and iris neovascularization.

### DETAILED DESCRIPTION OF THE INVENTION

In particular, this invention relates to the use of compounds of formula I: 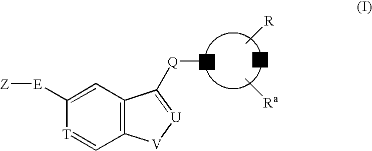
and pharmaceutically acceptable salts, prodrugs, and/or hydrates thereof,
wherein

Z represents an optionally substituted five-membered heteroaromatic ring selected from furan, thiophenyl, pyrrolyl, oxazolyl, thiazolyl, isoxazolyl, isothiazolyl, imidazolyl, pyrazolyl, oxadiazolyl, thiadiazolyl, triazolyl and tetrazolyl;

E represents a chemical bond or a straight or branched alkylene chain containing from 1 to 4 carbon atoms;

Q represents a straight or branched alkylene chain containing from 1 to 4 carbon atoms, optionally substituted in any position by a hydroxy group;

T represents nitrogen or CH;

U represents nitrogen or C—R²;

V represents oxygen, sulphur or N—R³;

R² and R³ independently represent hydrogen or C₁₋₆ alkyl;

M represents the residue of an azetidine, pyrrolidine or piperidine ring;

R represents a group of formula —W—R¹;

W represents a chemical bond or a straight or branched alkylene chain containing from 1 to 4 carbon atoms, optionally substituted in any position by a hydroxy group;

R¹ represents —OR^{x}, —SR^{x}, —SOR^{x}, —SO₂R^{x} or —NR^{x}R^{y};

R^{x} and R^{y} independently represent hydrogen, hydrocarbon or a heterocyclic group; or R^{x} and R^{y} together represent a C₂₋₆ alkylene group, which alkylene group may be optionally substituted by one or more substituents selected from C₁₋₆ alkyl, aryl and hydroxy, or fused with a phenyl ring; and

R^{a} represents hydrogen, hydroxy, hydrocarbon or a heterocyclic group.

The compounds of formula I above include those wherein T represents CH; W represents a chemical bond or a straight or branched alkylene chain containing from 1 to 4 carbon atoms; R¹ represents —OR^{x}, —SR^{x} or —NR^{x}R^{y}; R^{x} and R^{y} independently represent hydrogen, hydrocarbon or a heterocyclic group, or R^{x} and R^{y} together represent a C₂₋₆ alkylene group; and Z, E, Q, U, V, M and R^{a} are as defined above.

The present invention further relates to compounds of formula I above wherein Q represents a straight or branched alkylene chain containing from 1 to 4 carbon atoms; T represents CH; W represents a chemical bond or a straight or branched alkylene chain containing from 1 to 4 carbon atoms; R¹ represents —OR^{x}, —SR^{x} or —NR^{x}R^{y}; R^{x} and R^{y} independently represent hydrogen, hydrocarbon or a heterocyclic group, or R^{x} and R^{y} together represent a C₂₋₆ alkylene group; R^{a} represents hydrogen; and Z, E, U, V and M are as defined above.

The present invention still further relates to compounds of formula I above wherein Q represents a straight or branched alkylene chain containing from 1 to 4 carbon atoms; T represents nitrogen; U represents C—R²; V represents N—R³; W represents a chemical bond or a straight or branched alkylene chain containing from 1 to 4 carbon atoms; R¹ represents —OR^{x}, —SR^{x} or —NR^{x}R^{y}; R^{x} and R¹ independently represent hydrogen, hydrocarbon or a heterocyclic group, or R^{x} and R^{y} together represent a C₂₋₆ alkylene group; R¹ represents hydrogen; and Z, E, R², R³ and M are as defined above.

The five-membered heteroaromatic ring Z in the compounds of formula I above may be optionally substituted by one or, where possible, two substituents. As will be appreciated, where Z represents an oxadiazole, thiadiazole or tetrazole ring, only one substituent will be possible; otherwise, one or two optional substituents may be accommodated around the five-membered heteroaromatic ring Z. Examples of suitable substituents on the five-membered heteroaromatic ring Z include C₁₋₆ alkyl, C₂₋₆ alkenyl, C₂₋₆ alkynyl, C₃₋₇ cycloalkyl, aryl, aryl(C₁₋₆)alkyl, C₃₋₇ heterocycloalkyl, heteroaryl, heteroaryl(C₁₋₆)alkyl, C₁₋₆ alkoxy, C₁₋₆ alkylthio, amino, C₁₋₆ alkylamino, di(C₁₋₆)alkylamino, halogen, cyano or trifluoromethyl.

For use in medicine, the salts of the compounds of formula I will be pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulfuric acid, fumaric acid, maleic acid, succinic acid, acetic acid, benzoic acid, oxalic acid, citric acid, tartaric acid, carbonic acid or phosphoric acid. Furthermore, where the compounds of the invention carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts.

The term “hydrocarbon” as used herein includes straight-chained, branched and cyclic groups containing up to 18 carbon atoms, suitably up to 15 carbon atoms, and conveniently up to 12 carbon atoms. Suitable hydrocarbon groups include C₁₋₆ alkyl, C₂₋₆ alkenyl, C₂₋₆ alkynyl, C₃₋₇ cycloalkyl, C₃₋₇ cycloalkyl(C₁₋₆)alkyl, indanyl, aryl and aryl(C₁₋₆)alkyl.

The expression “a heterocyclic group” as used herein includes cyclic groups containing up to 18 carbon atoms and at least one heteroatom preferably selected from oxygen, nitrogen and sulphur. The heterocyclic group suitably contains up to 15 carbon atoms and conveniently up to 12 carbon atoms, and is preferably linked through carbon. Examples of suitable heterocyclic groups include C₃₋₇ heterocycloalkyl, C₃₋₇ heterocycloalkyl(C₁₋₆)alkyl, heteroaryl and heteroaryl(C₁₋₆)alkyl groups.

Suitable alkyl groups include straight-chained and branched alkyl groups containing from 1 to 6 carbon atoms. Typical examples include methyl and ethyl groups, and straight-chained or branched propyl, butyl and pentyl groups. Particular alkyl groups are methyl, ethyl, n-propyl, isopropyl, isobutyl, t-butyl and 2,2-dimethylpropyl.

Suitable alkenyl groups include straight-chained and branched alkenyl groups containing from 2 to 6 carbon atoms. Typical examples include vinyl, allyl and dimethylallyl groups.

Suitable alkynyl groups include straight-chained and branched alkynyl groups containing from 2 to 6 carbon atoms. Typical examples include ethynyl and propargyl groups.

Suitable cycloalkyl groups include groups containing from 3 to 7 carbon atoms. Particular cycloalkyl groups are cyclopropyl and cyclohexyl.

Typical examples of C₃₋₇ cycloalkyl(C₁₋₆)alkyl groups include cyclopropylmethyl, cyclohexylmethyl and cyclohexylethyl.

Particular indanyl groups include indan-1-yl and indan-2-yl.

Particular aryl groups include phenyl and naphthyl.

Particular aryl(C₁₋₆)alkyl groups include benzyl, phenylethyl, phenylpropyl and naphthylmethyl.

Suitable heterocycloalkyl groups include azetidinyl, pyrrolidyl, piperidyl, piperazinyl and morpholinyl groups.

Suitable heteroaryl groups include pyridyl, quinolyl, isoquinolyl, pyridazinyl, pyrimidinyl, pyrazinyl, pyranyl, furyl, benzofuryl, dibenzofuryl, thienyl, benzothienyl, pyrrolyl, indolyl, pyrazolyl, indazolyl, oxazolyl, isoxazolyl, thiazolyl, isothiazolyl, imidazolyl, benzimidazolyl, oxadiazolyl, thiadiazolyl, triazolyl and tetrazolyl groups.

The expression “heteroaryl(C₁₋₆)alkyl” as used herein includes furylmethyl, furylethyl, thienylmethyl, thienylethyl, oxazolylmethyl, oxazolylethyl, thiazolylmethyl, thiazolylethyl, imidazolylmethyl, imidazolylethyl, oxadiazolylmethyl, oxadiazolylethyl, thiadiazolylmethyl, thiadiazolylethyl, triazolylmethyl, triazolylethyl, tetrazolylmethyl, tetrazolylethyl, pyridylmethyl, pyridylethyl, pyrimidinylmethyl, pyrazinylmethyl, quinolylmethyl and isoquinolylmethyl.

The hydrocarbon and heterocyclic groups may in turn be optionally substituted by one or more groups selected from C₁₋₆ alkyl, adamantyl, phenyl, halogen, C₁₋₆ haloalkyl, C₁₋₆ aminoalkyl, trifluoromethyl, hydroxy, C₁₋₆ alkoxy, aryloxy, keto, C₁₋₃ alkylenedioxy, nitro, cyano, carboxy, C₂₋₆ alkoxycarbonyl, C₂₋₆ alkoxycarbonyl(C₁₋₆)alkyl, C₂₋₆ alkylcarbonyloxy, arylcarbonyloxy, aminocarbonyloxy, C₂₋₆ alkylcarbonyl, arylcarbonyl, C₁₋₆ alkylthio, C₁₋₆ alkylsulphinyl, C₁₋₆ alkylsulphonyl, arylsulphonyl, —NR^{v}R^{w}, —NR^{v}COR^{w}, —NR^{v}CO₂R^{w}, —NR^{v}SO₂R^{w}, —CH₂NR^{v}SO₂R^{w}, —NHCONR^{v}R^{w}, —CONR^{v}R^{w}, —SO₂NR^{v}R^{w} and —CH₂SO₂NR^{v}R^{w}, in which R^{v} and R^{w} independently represent hydrogen, C₁₋₆ alkyl, aryl or aryl(C₁₋₆)alkyl, or R^{v} and R^{w} together represent a C₂₋₆ alkylene group.

When R^{x} and R^{y}, or R^{v} and R^{w}, together represent a C₂₋₆ alkylene group, this group may be an ethylene, propylene, butylene, pentamethylene or hexamethylene group, preferably butylene or pentamethylene.

When R^{x} and R^{y} together represent a C₂₋₆ alkylene group, this group may be unsubstituted or substituted by one or more substituents selected from C₁₋₆ alkyl, aryl and hydroxy. Typical substituents include methyl, phenyl and hydroxy.

Furthermore, when R^{x} and R^{y} together represent a C₂₋₆ alkylene group, this group may optionally be fused with a phenyl ring. In this context, a typical group of formula —NR^{x}R^{y} as defined for the substituent R¹ is 1,2,3,4-tetrahydroisoquinolinyl.

The term “halogen” as used herein includes fluorine, chlorine, bromine and iodine, especially fluorine.

The present invention includes within its scope use of prodrugs of the compounds of formula I above. In general, such prodrugs will be functional derivatives of the compounds of formula I which are readily convertible in vivo into the required compound of formula I. Conventional procedures for the selection and preparation of suitable prodrug derivatives are described, for example, in Design of Prodrugs, ed. H. Bundgaard, Elsevier, 1985.

Where the compounds according to the invention have at least one asymmetric centre, they may accordingly exist as enantiomers. Where the compounds according to the invention possess two or more asymmetric centres, they may additionally exist as diastereoisomers. It is to be understood that all such isomers and mixtures thereof in any proportion are encompassed within the scope of the present invention.

In particular, where M represents the residue of a pyrrolidine ring, and the substituent R is attached to the 2-position thereof, then the absolute stereochemical configuration of the carbon atom at the point of attachment of the moiety R is preferably as depicted in structure IA as follows: 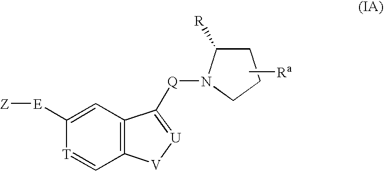
wherein Z, E, Q, T, U, V, R and R^{a} are as defined above.

Moreover, where M represents the residue of a pyrrolidine ring, and the substituent R is attached to the 3-position thereof, then the absolute stereochemical configuration of the carbon atom at the point of attachment of the moiety R is preferably as depicted in structure IB as follows: 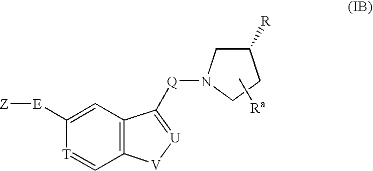
wherein Z, E, Q, T, U, V, R and R^{a} are as defined above.

The optionally substituted five-membered heteroaromatic ring Z in formula I is suitably a 1,3-oxazole, 1,3-thiazole, imidazole, 1,2,4-oxadiazole, 1,3,4-oxadiazole, 1,2,4-thiadiazole, 1,3,4-thiadiazole, 1,2,3-triazole, 1,2,4-triazole or tetrazole ring. Preferably, the ring is a 1,3-oxazole, 1,3-thiazole, imidazole, 1,2,4-oxadiazole, 1,2,4-thiadiazole or 1,2,4-triazole ring, in particular an imidazol-1-yl, 1,2,4-triazol-1-yl or 1,2,4-triazol-4-yl moiety.

Suitably, the five-membered heteroaromatic ring Z is unsubstituted. Examples of optional substituents which may typically be attached to the moiety Z include methyl, ethyl, benzyl and amino.

Where E, Q and W, which may be the same or different, represent straight or branched alkylene chains, these may be, for example, methylene, ethylene, 1-methylethylene, propylene, 2-methylpropylene or butylene. In addition, Q and W may be substituted in any position by a hydroxy group giving rise, for example, to a hydroxymethyl-methylene, 2-hydroxypropylene or 2-hydroxymethyl-propylene linkage. Moreover, E and W may each independently represent a chemical bond. Where E represents a chemical bond, the moiety Z is attached directly to the central fused bicyclic heteroaromatic ring system containing the variables T, U and V. Similarly, where W represents a chemical bond, the substituent R¹ is attached directly to the azetidine, pyrrolidine or piperidine ring of which M is the residue.

Suitably, E represents a chemical bond or a methylene linkage.

Suitably, Q represents an ethylene or propylene linkage.

The compound of formula I in accordance with the present invention is suitably an indole, benzofuran or benzthiophene derivative of formula IC, an indazole derivative of formula ID, or a pyrrolo[2,3-c]-pyridine derivative of formula IE: 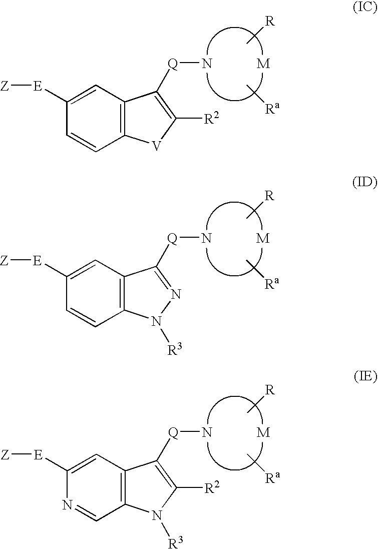
wherein Z, E, Q, V, M, R, R^{a}, R² and R³ are as defined above. Preferably, the compounds according to the invention are indole or pyrrolo[2,3-c]-pyridine derivatives of formula IF: 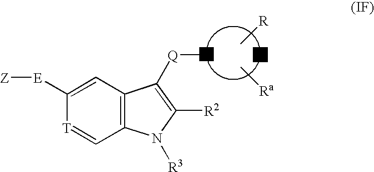
wherein Z, E, Q, T, M, R, R^{a}, R² and R³ are as defined above, in particular wherein R² and R³ are both hydrogen.

Suitably, W represents a chemical bond or a methylene or hydroxymethyl-methylene linkage, in particular a chemical bond or a methylene linkage.

Suitably, R^{x} and R^{y} independently represent hydrogen, C₁₋₆ alkyl, C₂₋₆ alkenyl, C₃₋₇ cycloalkyl(C₁₋₆)alkyl, indanyl, aryl, aryl(C₁₋₆)alkyl, heteroaryl or heteroaryl(C₁₋₆)alkyl, any of which groups may be optionally substituted by one or more substituents selected typically from C₁₋₆ alkyl, halogen, hydroxy, C₁₋₆ alkoxy, aminocarbonyloxy, amino, C₂₋₆ alkylcarbonylamino, C₁₋₆ alkylsulphonylamino and C₁₋₆ alkylaminosulphonylmethyl. Particular values of R^{x} and R^{y} include hydrogen, methyl, hydroxyethyl, isobutyl, 2,2-dimethylpropyl, allyl, dimethylallyl, 1-cyclohexylethyl, 2-cyclohexylethyl, indanyl, hydroxy-indanyl, phenyl, benzyl, methyl-benzyl, fluorobenzyl, methoxybenzyl, acetylamino-benzyl, 1-phenylethyl, 2-phenylethyl, 2-hydroxy-1-phenylethyl, 2-methoxy-1-phenylethyl, 2-aminocarbonyloxy-1-phenylethyl, 1-(fluorophenyl)ethyl, 1-(fluorophenyl)-2-hydroxyethyl, 1-(fluorophenyl)-2-methoxyethyl, 1-(acetylamino-phenyl)ethyl, 2-(acetylaminophenyl)ethyl, 2-hydroxy-1-phenylprop-1-yl, 1-phenylprop-2-yl, 2-phenylprop-2-yl, 1-hydroxy-1-phenylprop-2-yl, 1-hydroxy-2-phenylprop-2-yl, 1-hydroxy-3-phenylprop-2-yl, furylmethyl, thienylmethyl and pyridylmethyl.

In addition, where R^{x} and R^{y} together represent an optionally substituted or phenyl ring-fused C₂₋₆ alkylene group, the substituent —NR^{x}R^{y} as defined for R¹ may suitably represent 3,3-dimethylpiperidinyl, 2-phenylpiperidinyl, 3-hydroxy-2-phenylpiperidinyl or 1,2,3,4-tetrahydroisoquinolin-2-yl.

Suitable values for the substituent R¹ include hydroxy, benzyloxy, methoxy-benzyloxy, pyridylmethoxy, benzylthio, fluorobenzyl-thio, phenylsulphinyl, benzylsulphinyl, fluorobenzyl-sulphinyl, fluorobenzyl-sulphonyl, amino, methylamino, indanylamino, hydroxyindanyl-amino, benzylamino, N-(methylbenzyl)-amino, N-(acetylamino-benzyl)-amino, N-(1-phenylethyl)-amino, N-(2-phenylethyl)-amino, N-(2-hydroxy-1-phenylethyl)-amino, N-(2-methoxy-1-phenylethyl)-amino, N-(2-aminocarbonyloxy-1-phenylethyl)-amino, N-[1-(fluorophenyl)ethyl]-amino, N-[1-(fluorophenyl)-2-hydroxyethyl]-amino, N-[1-(fluorophenyl)-2-methoxyethyl]-amino, N-[1-(acetylamino-phenyl)ethyl]-amino, N-[2-(acetylamino-phenyl)ethyl]-amino, N-(2-hydroxy-1-phenylprop-1-yl)-amino, N-(1-phenylprop-2-yl)-amino, N-(2-phenylprop-2-yl)-amino, N-(1-hydroxy-1-phenylprop-2-yl)-amino, N-(1-hydroxy-2-phenylprop-2-yl)-amino, N-(1-hydroxy-3-phenylprop-2-yl)-amino, N-(furylmethyl)-amino, N-(pyridylmethyl)-amino, dimethylamino, N-isobutyl-N-methylamino, N-(2,2-dimethylpropyl)-N-methylamino, N-allyl-N-methylamino, N-(3,3-dimethylprop-2-en-1-yl)-N-methylamino, N-(1-cyclohexylethyl)-N-methylamino, N-benzyl-N-methylamino, N-methyl-N-(methylbenzyl)-amino, N-(fluorobenzyl)-N-methylamino, N-(acetylamino-benzyl)-N-methylamino, N-methyl-N-(1-phenylethyl)-amino, N-methyl-N-(2-phenylethyl)-amino, N-(2-hydroxy-1-phenylethyl)-N-methylamino, N-(2-methoxy-1-phenylethyl)-N-methylamino, N-[2-(acetylamino-phenyl)ethyl]-N-methylamino, N-(furylmethyl)-N-methylamino, N-methyl-N-(thienylmethyl)-amino, N-benzyl-N-(2-hydroxyethyl)-amino, N,N-bis(furylmethyl)-amino, 3,3-dimethylpiperidinyl, 2-phenylpiperidinyl, 3-hydroxy-2-phenylpiperidinyl and 1,2,3,4-tetrahydroisoquinolin-2-yl.

Particular values of the group R include hydroxy, benzyloxy, benzyloxymethyl, methoxy-benzyloxy, pyridylmethoxy, benzylthio-methyl, fluorobenzylthio-methyl, phenylsulphinylmethyl, benzylsulphinylmethyl, fluorobenzyl-sulphinyl, fluorobenzyl-sulphinylmethyl, fluorobenzyl-sulphonylmethyl, indanylamino, indanylaminomethyl, hydroxyindanyl-amino, benzylamino, benzylaminomethyl, 1-(N-benzylamino)-2-hydroxyethyl, N-(methylbenzyl)-aminomethyl, N-(acetylamino-benzyl)-amino, N-(acetylamino-benzyl)-aminomethyl, N-(1-phenylethyl)-amino, N-(1-phenylethyl)-aminomethyl, N-(2-phenylethyl)-aminomethyl, N-(2-hydroxy-1-phenylethyl)-amino, N-(2-hydroxy-1-phenylethyl)-aminomethyl, N-(2-methoxy-1-phenylethyl)-amino, N-(2-aminocarbonyloxy-1-phenylethyl)-amino, N-[1-(fluorophenyl)ethyl]-amino, N-[1-(fluorophenyl)-2-hydroxyethyl]-amino, N-[1-(fluorophenyl)-2-methoxyethyl]-amino, N-[1-(acetylamino-phenyl)ethyl]-amino, N-[1-(acetylaminophenyl)ethyl]-aminomethyl, N-[2-(acetylamino-phenyl)ethyl]-amino, N-(2-hydroxy-1-phenylprop-1-yl)-amino, N-(1-phenylprop-2-yl)-amino, N-(2-phenylprop-2-yl)-aminomethyl, N-(1-hydroxy-1-phenylprop-2-yl)-amino, N-(1-hydroxy-2-phenylprop-2-yl)-amino, N-(1-hydroxy-3-phenylprop-2-yl)-amino, N-(furylmethyl)-amino, N-(furylmethyl)-aminomethyl, N-(pyridylmethyl)-aminomethyl, N-isobutyl-N-methyl-aminomethyl, N-(2,2-dimethylpropyl)-N-methyl-aminomethyl, N-allyl-N-methylamino, N-(3,3-dimethylprop-2-en-1-yl)-N-methylamino, N-(1-cyclohexylethyl)-N-methyl-aminomethyl, N-benzyl-N-methylamino, N-benzyl-N-methyl-aminomethyl, N-methyl-N-(methylbenzyl)-aminomethyl, N-(fluorobenzyl)-N-methylamino, N-(acetylamino-benzyl)-N-methyl-aminomethyl, N-methyl-N-(1-phenylethyl)-aminomethyl, N-methyl-N-(2-phenylethyl)-aminomethyl, N-(2-hydroxy-1-phenylethyl)-N-methylamino, N-(2-hydroxy-1-phenylethyl)-N-methyl-aminomethyl, N-(2-methoxy-1-phenylethyl)-N-methylamino, N-[2-(acetylamino-phenyl)ethyl]-N-methylamino, N-(furylmethyl)-N-methylamino, N-methyl-N-(thienylmethyl)-amino, N-benzyl-N-(2-hydroxyethyl-aminomethyl, N,N-bis(furylmethyl)-amino, 3,3-dimethylpiperidinylmethyl, 2-phenylpiperidinyl, 2-phenylpiperidinylmethyl, 3-hydroxy-2-phenylpiperidinylmethyl and 1,2,3,4-tetrahydroisoquinolin-2-yl.

Suitable values of R^{a} include hydrogen, hydroxy and benzyl, especially hydrogen.

Suitably, R² and R³ independently represent hydrogen or methyl, especially hydrogen.

A particular sub-class of compounds useful to the invention is represented by the compounds of formula IIA, and pharmaceutically acceptable salts and prodrugs thereof: 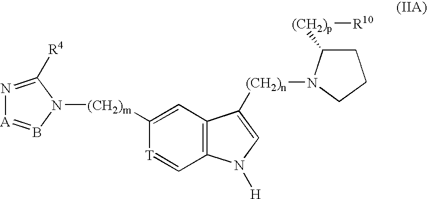
wherein

m is zero, 1, 2 or 3, preferably zero or 1;

n is 2, 3 or 4, preferably 2 or 3;

p is zero, 1 or 2;

T represents nitrogen or CH;

A represents nitrogen or CH;

B represents nitrogen or C—R⁵;

R⁴ and R⁵ independently represent hydrogen, C₁₋₆ alkyl, C₂₋₆ alkenyl, C₃₋₇ cycloalkyl, aryl, aryl(C₁₋₆)alkyl, C₃₋₇ heterocycloalkyl, heteroaryl, heteroaryl(C₁₋₆)alkyl, C₁₋₆ alkoxy, C₁₋₆ alkylthio, amino, C₁₋₆ alkylamino, di(C₁₋₆)alkylamino, halogen, cyano or trifluoromethyl; and

R¹⁰ represents —X—R¹¹ or a group of formula (a) or (b): 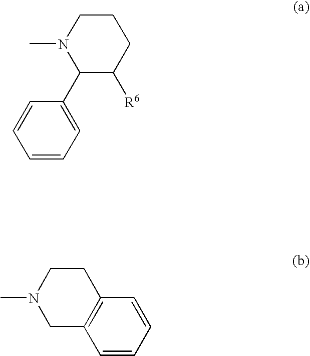
in which

R⁶ represents hydrogen or hydroxy;

X represents oxygen, sulphur, —SO—, —SO₂— or N—R¹²; and

R¹¹ and R¹² independently represent hydrogen, C₁₋₆ alkyl, C₂₋₆ alkenyl, C₃₋₇ cycloalkyl(C₁₋₆)alkyl, indanyl, aryl, aryl(C₁₋₆)alkyl, heteroaryl or heteroaryl(C₁₋₆)alkyl, any of which groups may be optionally substituted.

Examples of suitable optional substituents on the groups R¹¹ and R¹² include C₁₋₆ alkyl, halogen, cyano, trifluoromethyl, hydroxy, C₁₋₆ alkoxy, aminocarbonyloxy, C₂₋₆ alkylcarbonyl, amino, C₁₋₆ alkylamino, di(C₁₋₆)alkylamino, C₂₋₆ alkylcarbonylamino, C₁₋₆ alkylsulphonylamino and C₁₋₆ alkylaminosulphonylmethyl.

Particular values of R⁴ and R⁵ include hydrogen, methyl, ethyl, benzyl and amino, especially hydrogen.

Particular values of R¹¹ and R¹² include hydrogen, methyl, hydroxyethyl, isobutyl, 2,2-dimethylpropyl, allyl, dimethylallyl, 1-cyclohexylethyl, 2-cyclohexylethyl, indanyl, hydroxy-indanyl, phenyl, benzyl, methyl-benzyl, fluorobenzyl, methoxy-benzyl, acetylamino-benzyl, 1-phenylethyl, 2-phenylethyl, 2-hydroxy-1-phenylethyl, 2-methoxy-1-phenylethyl, 2-aminocarbonyloxy-1-phenylethyl, 1-(fluorophenyl)ethyl, 1-(fluorophenyl)-2-hydroxyethyl, 1-(fluorophenyl)-2-methoxyethyl, 1-(acetylaminophenyl)ethyl, 2-(acetylamino-phenyl)ethyl, 2-hydroxy-1-phenylprop-1-yl, 1-phenylprop-2-yl, 2-phenylprop-2-yl, 1-hydroxy-1-phenylprop-2-yl, 1-hydroxy-2-phenylprop-2-yl, 1-hydroxy-3-phenylprop-2-yl, furylmethyl, thienylmethyl and pyridylmethyl.

In relation to formula IIA, the variable p is preferably 1.

Another sub-class of compounds according to the invention is represented by the compounds of formula IIB, and salts and prodrugs thereof: 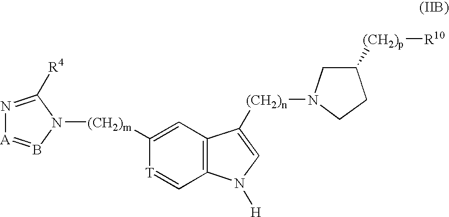
wherein m, n, p, T, A, B, R⁴ and R¹⁰ are as defined with reference to formula IIA above.

In relation to formula IIB, the variable p is suitably zero or 1.

A further sub-class of compounds according to the invention is represented by the compounds of formula IIC, and salts and prodrugs thereof: 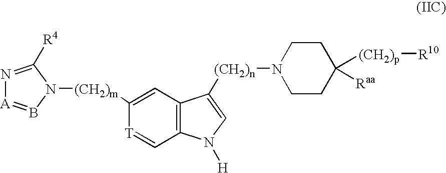
wherein

R^{aa} represents hydrogen, hydroxy or aryl(C₁₋₆)alkyl; and

m, n, p, T, A, B, R⁴ and R¹⁰ are as defined with reference to formula IIA above.

Suitable values of R^{aa} include hydrogen, hydroxy and benzyl, especially hydrogen.

In relation to formula IIC, the variable p is suitably zero or 1.

In one subset of the compounds of formula IIC above, R^{aa} is hydrogen.

A still further sub-class of compounds according to the invention is represented by the compounds of formula IID, and salts and prodrugs thereof: 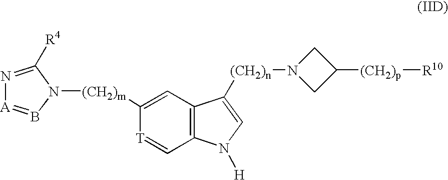
wherein m, n, p, T, A, B, R⁴ and R¹⁰ are as defined with reference to formula IIA above.

In relation to formula IID, the variable p is suitably zero or 1.

The present invention also includes compounds of formula IIA, IIB, IIC and IID as defined above wherein T represents CH; R¹⁰ represents —X—R¹¹; X represents oxygen, sulphur or N—R¹²; R¹¹ and R¹² independently represent hydrogen, C₁₋₆ alkyl, aryl, aryl(C₁₋₆)alkyl, heteroaryl or heteroaryl(C₁₋₆)alkyl, any of which groups may be optionally substituted; and m, n, p, A, B and R⁴ are as defined above.

The present invention further includes use of compounds of formula IIA, IIB and IIC as defined above wherein T represents nitrogen; R¹⁰ represents —X—R¹¹; X represents oxygen, sulphur or N—R¹²; R¹¹ and R¹² independently represent hydrogen, C₁₋₆ alkyl, aryl, aryl(C₁₋₆)alkyl, heteroaryl or heteroaryl(C₁₋₆)alkyl, any of which groups may be optionally substituted; R^{aa} represents hydrogen; and m, n, p, A, B and R⁴ are as defined above.

Compounds useful in the invention are:
(3R)-3-benzyloxy-1-[2-(5-(1,2,4-triazol-4-yl)-1H-indol-3-yl)ethyl]pyrrolidine;
(3R)-3-(4-methoxyphenyl)methoxy-1-[2-(5-(1,2,4-triazol-4-yl)-1H-indol-3-yl)ethyl]pyrrolidine;
(3R)-3-benzyloxymethyl-1-[2-(5-(1,2,4-triazol-4-yl)-1H-indol-3-yl)ethyl]pyrrolidine;
(3S)-3-(N-benzyl-N-methyl)aminomethyl-1-[2-(5-(1,2,4-triazol-4-yl)-1H-indol-3-yl)ethyl]pyrrolidine;
(2S)-2-(N-benzyl-N-methyl)aminomethyl-1-[2-(5-(1,2,4-triazol-4-yl)-1H-indol-3-yl)ethyl]pyrrolidine;
(3S)-3-(N-benzyl)aminomethyl-1-[2-(5-(1,2,4-triazol-4-yl)-1H-indol-3-yl)ethyl]pyrrolidine;
4-(4-acetylaminophenyl)methylamino-1-[3-(5-(1,2,4-triazol-4-yl)-1H-indol-3-yl)propyl]piperidine;
4-benzylamino-1-[3-(5-(1,2,4-triazol-4-yl)-1H-indol-3-yl)propyl]piperidine;
4-(N-benzyl-N-methyl)amino-1-[3-(5-(1,2,4-triazol-4-yl)-1H-indol-3-yl)propyl]piperidine;
4-(N-benzyl)aminomethyl-1-[3-(5-(1,2,4-triazol-4-yl)-1H-indol-3-yl)propyl]piperidine;
(2S)-2-(N-benzyl-N-methylaminomethyl)-1-[2-(5-(1,2,4-triazol-1-yl)-1H-pyrrolo[2,3-c]pyridin-3-yl)ethyl]pyrrolidine;
4-(N-benzyl-N-methyl)aminomethyl-1-[3-(5-(1,2,4-triazol-4-yl)-1H-indol-3-yl)propyl]piperidine;
1-[3-(5-(1,2,4-triazol-4-yl)-1H-indol-3-yl)propyl]-4-[(R)-α-(methyl)benzylamino]piperidine;
1-[3-(5-(1,2,4-triazol-4-yl)-1H-indol-3-yl)propyl]-4-[(S)-α-(methyl)benzylamino]piperidine;
1-[3-(5-(1,2,4-triazol-4-yl)-1H-indol-3-yl)propyl]-4-[(S)-α-(hydroxymethyl)benzylamino]piperidine;
1-[3-(5-(1,2,4-triazol-4-yl)-1H-indol-3-yl)propyl]-4-[(R)-α-(hydroxymethyl)benzylamino]piperidine;
1-[3-(5-(1,2,4-triazol-4-yl)-1H-indol-3-yl)propyl]-4-[(S)-(1-hydroxymethyl-2-phenyl)ethylamino]piperidine;
1-[3-(5-(1,2,4-triazol-4-yl)-1H-indol-3-yl)propyl]-4-[(1R,2S)-(2-hydroxy-1-methyl-2-phenyl)ethylamino]piperidine;
1-[3-(5-(1,2,4-triazol-4-yl)-1H-indol-3-yl)propyl]-4-[(1S,2R)-(2-hydroxy-1-methyl-2-phenyl)ethylamino]piperidine;
1-[3-(5-(1,2,4-triazol-4-yl)-1H-indol-3-yl)propyl]-4-[(1R,2R)-(2-hydroxy-1-methyl-2-phenyl)ethylamino]piperidine;
1-[3-(5-(1,2,4-triazol-4-yl)-1H-indol-3-yl)propyl]-4-[2-(4-acetylaminophenyl)ethylamino]piperidine;
1-[3-(5-(1,2,4-triazol-4-yl)-1H-indol-3-yl)propyl]-4-[(R)-α-(methyl)benzylamino]methylpiperidine;
1-[3-(5-(1,2,4-triazol-4-yl)-1H-indol-3-yl)propyl]-4-[(S)-α-(methyl)benzylamino]methylpiperidine;
1-[3-(5-(1,2,4-triazol-4-yl)-1H-indol-3-yl)propyl]-4-[(S)-1-(4-acetylaminophenyl)ethylamino]methylpiperidine;
1-[3-(5-(1,2,4-triazol-4-yl)-1H-indol-3-yl)propyl]-4-[(R)-1-(4-acetylaminophenyl)ethylamino]methylpiperidine;
1-[3-(5-(1,2,4-triazol-4-yl)-1H-indol-3-yl)propyl]-4-[N—[(R)-α-(hydroxymethyl)benzyl]-N-methylamino]piperidine;
1-[3-(5-(1,2,4-triazol-4-yl)-1H-indol-3-yl)propyl]-4-[N—[(S)-α-(hydroxymethyl)benzyl]-N-methylamino]piperidine;
1-[3-(5-(1,2,4-triazol-4-yl)-1H-indol-3-yl)propyl]-4-[N-(2-(4-acetylaminophenyl)ethyl)-N-methylamino]piperidine;
1-[3-(5-(1,2,4-triazol-4-yl)-1H-indol-3-yl)propyl]-4-[N-(4-acetylaminobenzyl)-N-methylamino]methyl piperidine;
1-[3-(5-(1,2,4-triazol-4-yl)-1H-indol-3-yl)propyl]-4-[(R)-α-(hydroxymethyl)benzylamino]methyl piperidine;
(3S)-3-(4-acetylaminobenzyl)aminomethyl-1-[2-(5-(1,2,4-triazol-4-yl)-1H-indol-3-yl)ethyl]pyrrolidine;
(3R)-3-(N-benzyl)aminomethyl-1-[2-(5-(1,2,4-triazol-4-yl)-1H-indol-3-yl)ethyl]pyrrolidine;
3-(N-benzyl)aminomethyl-1-[2-(5-(1,2,4-triazol-4-yl)-1H-indol-3-yl)ethyl]azetidine;
4-benzyl-4-hydroxy-1-[3-(5-(1,2,4-triazol-4-yl)-1H-indol-3-yl)propyl]piperidine;
3-(N-benzyl)aminomethyl-1-[3-(5-(1,2,4-triazol-4-yl)-1H-indol-3-yl)propyl]azetidine;
4-(N-benzyl)aminomethyl-4-hydroxy-1-[3-(5-(1,2,4-triazol-4-yl)-1H-indol-3-yl)propyl]piperidine;
4-(N-benzyl-N-methyl)aminomethyl-4-hydroxy-1-[3-(5-(1,2,4-triazol-4-yl)-1H-indol-3-yl)propyl]piperidine;
3-(N-benzyl-N-methyl)aminomethyl-1-[2-(5-(1,2,4-triazol-4-yl)-1H-indol-3-yl)ethyl]azetidine;
(3S)-3-[N—(R)-α-(methyl)benzyl]aminomethyl-1-[2-(5-(1,2,4-triazol-4-yl)-1H-indol-3-yl)ethyl]pyrrolidine;
(3S)-3-[N—(S)-α-(methyl)benzyl]aminomethyl-1-[2-(5-(1,2,4-triazol-4-yl)-1H-indol-3-yl)ethyl]pyrrolidine;
(3S)-3-[N—(R)-α-(hydroxymethyl)benzyl]aminomethyl-1-[2-(5-(1,2,4-triazol-4-yl)-1H-indol-3-yl)ethyl]pyrrolidine;
(3S)-3-[N—(S)-α-(hydroxymethyl)benzyl]aminomethyl-1-[2-(5-(1,2,4-triazol-4-yl)-1H-indol-3-yl)ethyl]pyrrolidine;
(3S)-3-[N-benzyl-N-(2-hydroxy)ethyl]aminomethyl-1-[2-(5-(1,2,4-triazol-4-yl)-1H-indol-3-yl)ethyl]pyrrolidine;
(3S)-3-[N-(2-phenylethyl)amino]methyl-1-[2-(5-(1,2,4-triazol-4-yl)-1H-indol-3-yl)ethyl]pyrrolidine;
(3S)-3-[N-(2-phenylethyl)-N-methylamino]methyl-1-[2-(5-(1,2,4-triazol-4-yl)-L H-indol-3-yl)ethyl]pyrrolidine;
(3S)-3-(N-α-dimethylbenzyl)aminomethyl-1-[2-(5-(1,2,4-triazol-4-yl)-1H-indol-3-yl)ethyl]pyrrolidine;
(3S)-3-[N—(S)-α-methylbenzyl]aminomethyl-1-[2-(5-(1,2,4-triazol-1-yl)-1H-indol-3-yl)ethyl]pyrrolidine;
(3S)-3-[N—(R)-α-(hydroxymethyl)benzyl]aminomethyl-1-[2-(5-(1,2,4-triazol-1-yl)-1H-indol-3-yl)ethyl]pyrrolidine;
(3S)-3-(N-benzyl)aminomethyl-1-[2-(5-(1,2,4-triazol-1-ylmethyl)-1H-indol-3-yl)ethyl]pyrrolidine;
(3S)-3-[N—(S)-α-methylbenzyl]aminomethyl-[2-(5-(1,2,4-triazol-1-ylmethyl)-1H-indol-3-yl)ethyl]pyrrolidine;
(3S)-3-[N—(R)-α-(hydroxymethyl)benzyl]aminomethyl-1-[2-(S-(1,2,4-triazol-1-ylmethyl)-1H-indol-3-yl)ethyl]pyrrolidine;
(3S)-3-(N-benzyl-N-methyl)aminomethyl-1-[2-(5-(imidazol-1-yl)-1H-indol-3-yl)ethyl]pyrrolidine;
(3S)-3-(N-benzyl-N-methyl)aminomethyl-1-[2-(5-(1,2,4-triazol-1-ylmethyl)-1H-indol-3-yl)ethyl]pyrrolidine;
(3R)-3-[N-methyl-N—(S)-α-methylbenzyl]aminomethyl-1-[2-(5-(1,2,4-triazol-1-ylmethyl)-1H-indol-3-yl)ethyl]pyrrolidine;
(3R)-3-[N-methyl-N—(R)-α-hydroxymethylbenzyl]aminomethyl-1-[2-(5-(1,2,4-triazol-1-ylmethyl)-1H-indol-3-yl)ethyl]pyrrolidine;
(3R)-3-[N-methyl-N—(S)-α-methylcyclohexylmethyl]aminomethyl-1-[2-(5-(1,2,4-triazol-1-ylmethyl)-1H-indol-3-yl)ethyl]pyrrolidine;
(3R)-3-[3-(R)-hydroxy-2-(R)-phenylpiperidin-1-yl]methyl-1-[2-(5-(1,2,4-triazol-1-ylmethyl)-1H-indol-3-yl)ethyl]pyrrolidine;
(3R)-3-[3-(R)-hydroxy-2-(R)-phenylpiperidin-1-yl]methyl-1-[2-(5-(1,2,4-triazol-1-yl)-1H-indol-3-yl)ethyl]pyrrolidine;
4-hydroxy-4-(phenylsulfinyl)methyl-1-[3-(5-(1,2,4-triazol-4-yl)-1H-indol-3-yl)propyl]piperidine;
(3R)-3-[2-(R,S)-phenylpiperidin-1-yl]methyl-1-[2-(5-(1,2,4-triazol-1-ylmethyl)-1H-indol-3-yl)ethyl]pyrrolidine;
4-(3,3-dimethylpiperidin-1-yl)methyl-4-hydroxy-1-[3-(5-(1,2,4-triazol-4-yl)-1H-indol-3-yl)propyl]piperidine;
4-hydroxy-4-(1,2,3,4-tetrahydroisoquinolin-2-yl)methyl-1-[3-(5-(1,2,4-triazol-4-yl)-1H-indol-3-yl)propyl]piperidine;
4-hydroxy-4-(N-isobutyl-N-methyl)aminomethyl-1-[3-(5-(1,2,4-triazol-4-yl)-1H-indol-3-yl)propyl]piperidine;
4-[N-benzyl-N-(2-hydroxyethyl)amino]methyl-4-hydroxy-1-[3-(5-(1,2,4-triazol-4-yl)-1H-indol-3-yl)propyl]piperidine;
4-[N-(2,2-dimethylpropyl)-N-methylamino]methyl-4-hydroxy-1-[3-(5-(1,2,4-triazol-4-yl)-1H-indol-3-yl)propyl]piperidine;
4-[N—(R)-α-hydroxymethylbenzyl-N-methylamino]methyl-4-hydroxy-1-[3-(5-(1,2,4-triazol-4-yl)-1H-indol-3-yl)propyl]piperidine;
4-hydroxy-4-(2-methylphenylmethyl)aminomethyl-1-[3-(5-(1,2,4-triazol-4-yl)-1H-indol-3-yl)propyl]piperidine;
4-hydroxy-4-[N-(2-methylphenylmethyl)-N-methylamino]methyl-1-[3-(5-(1,2,4-triazol-4-yl)-1H-indol-3-yl)propyl]piperidine;
3-(benzylamino)methyl-3-hydroxy-1-[3-(5-(1,2,4-triazol-4-yl)-1H-indol-3-yl)propyl]pyrrolidine;
3-(benzylamino)methyl-3-hydroxy-1-[2-(5-(1,2,4-triazol-4-yl)-1H-indol-3-yl)ethyl]pyrrolidine;
1-[3-(5-(1,2,4-triazol-4-yl)-1H-indol-3-yl)propyl]-4-[(R)-α-(carbamoyl-oxymethyl)benzylamino]piperidine;
1-[3-(5-(1,2,4-triazol-4-yl)-1H-indol-3-yl)propyl]-4-[(1R,2S)-2-hydroxy-1-phenylpropylamino]piperidine;
1-[3-(5-(1,2,4-triazol-4-yl)-1H-indol-3-yl)propyl]-4-[(1R,2R)-2-hydroxy-1-phenylpropylamino]piperidine;
1-[3-(5-(1,2,4-triazol-4-yl)-1H-indol-3-yl)propyl]-4-[(R,S)-1-hydroxy-2-phenylprop-2-ylamino]piperidine;
1-[3-(5-(1,2,4-triazol-4-yl)-1H-indol-3-yl)propyl]-4-[(R)-2-hydroxy-1-(4-fluorophenyl)ethylamino]piperidine;
1-[3-(5-(1,2,4-triazol-4-yl)-1H-indol-3-yl)propyl]-4-[(1R,2R)-2-hydroxyindan-1-ylamino)piperidine;
1-[3-(5-(1,2,4-triazol-4-yl)-1H-indol-3-yl)propyl]-4-[(R,S)-indan-1-ylamino]piperidine;
1-[3-(5-(1,2,4-triazol-4-yl)-1H-indol-3-yl)propyl]-4-[(R,S)-1-(4-fluorophenyl)ethylamino]piperidine;
1-[3-(5-(1,2,4-triazol-4-yl)-1H-indol-3-yl)propyl]-4-[(R)-1-phenylprop-2-ylamino]piperidine;
1-[3-(5-(1,2,4-triazol-4-yl)-1H-indol-3-yl)propyl]-4-[N-(3,3-dimethylallyl)-N-methylamino]piperidine;
1-[3-(5-(1,2,4-triazol-4-yl)-1H-indol-3-yl)propyl]-4-(N-allyl-N-methylamino)piperidine;
1-[3-(5-(1,2,4-triazol-4-yl)-1H-indol-3-yl)propyl]-4-(indan-1-ylaminomethyl)piperidine;
1-[3-(5-(1,2,4-triazol-4-yl)-1H-indol-3-yl)propyl]-4-[N—(R)-α-(hydroxymethyl)benzyl-N-methylaminomethyl]piperidine;
(3R)-3-(benzylthio)methyl-1-[2-(5-(1,2,4-triazol-4-yl)-1H-indol-3-yl)ethyl]pyrrolidine;
(±)-1-[3-(5-(1,2,4-triazol-4-yl)-1H-indol-3-yl)propyl]-4-(1-benzylamino-2-hydroxyethyl)piperidine;
1-[3-(5-(1,2,4-triazol-1-yl)-1H-indol-3-yl)propyl]-4-[(R)-1-(hydroxymethyl)benzylamino]piperidine;
1-[3-(5-(imidazol-1-yl)-1H-indol-3-yl)propyl]-4-[(R)-α-(methyl)benzylamino]piperidine;
1-[3-(5-(imidazol-1-yl)-1H-indol-3-yl)propyl]-4-[(R)-α-(hydroxymethyl)benzylamino]piperidine;
1-[3-(5-(1,2,4-triazol-1-ylmethyl)-1H-indol-3-yl)propyl]-4-[(R)-α-(hydroxymethyl)benzylamino]piperidine;
1-[3-(5-(1,2,4-triazol-4-yl)-1H-indol-3-yl)propyl]1-4-[(R)-α-(methoxymethyl)benzylamino]piperidine;
1-[3-(5-(1,2,4-triazol-4-yl)-1H-indol-3-yl)propyl]-4-[N—(R)-α-(methoxymethyl)benzyl-N-methylamino]piperidine;
1-[3-(5-(imidazol-1-yl)-1H-indol-3-yl)propyl]-4-[(R)-α-(methoxymethyl)benzylamino]piperidine;
1-[3-(5-(1,2,4-triazol-1-ylmethyl)-1H-indol-3-yl)propyl]-4-[(R)-1-(4-fluorophenyl)-2-methoxyethylamino]piperidine;
1-[3-(5-(1,2,4-triazol-1-ylmethyl)-1H-indol-3-yl)propyl]-4-[N-(4-fluorobenzyl)-N-methylamino]piperidine;
1-[3-(5-(1,2,4-triazol-4-yl)-1H-indol-3-yl)propyl]-4-(2-phenylpiperidin-1-yl)piperidine;
1-[3-(5-(1,2,4-triazol-4-yl)-1H-indol-3-yl)propyl]-4-[(R)-1-(4-fluorophenyl)-2-methoxyethylamino]piperidine;
(3R)-3-(benzylsulfinyl)methyl-1-[2-(5-(1,2,4-triazol-4-yl)-1H-indol-3-yl)ethyl]pyrrolidine;
(3R)-3-(4-fluorobenzylthio)methyl-1-[2-(5-(1,2,4-triazol-1-ylmethyl)-1H-indol-3-yl)ethyl]pyrrolidine;
(3R)-3-(4-fluorobenzylsulfinyl)methyl-1-[2-(5-(1,2,4-triazol-1-ylmethyl)-1H-indol-3-yl)ethyl]pyrrolidine;
(3R)-3-(4-fluorobenzylsulfonyl)methyl-1-[2-(5-(1,2,4-triazol-1-ylmethyl)-1H-indol-3-yl)ethyl]pyrrolidine;
4-(4-fluorobenzylsulfinyl)-1-[3-(5-(1,2,4-triazol-4-yl)-1H-indol-3-yl)propyl]piperidine; and salts and prodrugs thereof.

Preferred compounds for use in this invention are:
(3S)-3-[N-(2-phenylethyl)amino]methyl-1-[2-(5-(1,2,4-triazol-4-yl)-1H-indol-3-yl)ethyl]pyrrolidine;

(3S)-3-[N-(2-phenylethyl)-N-methylamino]methyl-1-[2-(5-(1,2,4-triazol-4-yl)-1H-indol-3-yl)ethyl]pyrrolidine;
(3S)-3-(N-α-dimethylbenzyl)aminomethyl-1-[2-(5-(1,2,4-triazol-4-yl)-1H-indol-3-yl)ethyl]pyrrolidine;
(3S)-3-[N—(S)-α-methylbenzyl]aminomethyl-1-[2-(5-(1,2,4-triazol-1-yl)-1H-indol-3-yl)ethyl]pyrrolidine;
(3S)-3-[N—(R)-α-(hydroxymethyl)benzyl]aminomethyl-1-[2-(5-(1,2,4-triazol-1-yl)-1H-indol-3-yl)ethyl]pyrrolidine;
(3S)-3-(N-benzyl)aminomethyl-1-[2-(5-(1,2,4-triazol-1-ylmethyl)-1H-indol-3-yl)ethyl]pyrrolidine;
(3S)-3-[N—(S)-α-methylbenzyl]aminomethyl-1-[2-(5-(1,2,4-triazol-1-ylmethyl)-1H-indol-3-yl)ethyl]pyrrolidine;
(3S)-3-[N—(R)-α-(hydroxymethyl)benzyl]aminomethyl-1-[2-(5-(1,2,4-triazol-1-ylmethyl)-1H-indol-3-yl)ethyl]pyrrolidine;
(3S)-3-(N-benzyl-N-methyl)aminomethyl-1-[2-(5-(imidazol-1-yl)-1H-indol-3-yl)ethyl]pyrrolidine;
(3S)-3-(N-benzyl-N-methyl)aminomethyl-1-[2-(5-(1,2,4-triazol-1-ylmethyl)-1H-indol-3-yl)ethyl]pyrrolidine; and pharmaceutically acceptable salts, prodrugs, and/or hydrates thereof.

More preferred compounds for use in this invention are:
(3S)-3-[N-(2-phenylethyl)amino]methyl-1-[2-(5-(1,2,4-triazol-4-yl)-1H-indol-3-yl)ethyl]pyrrolidine;
(3S)-3-[N-(2-phenylethyl)-N-methylamino]methyl-1-[2-(5-(1,2,4-triazol-4-yl)-1H-indol-3-yl)ethyl]pyrrolidine;
(3S)-3-(N-α-dimethylbenzyl)aminomethyl-1-[2-(5-(1,2,4-triazol-4-yl)-1H-indol-3-yl)ethyl]pyrrolidine;
(3S)-3-[N—(S)-α-methylbenzyl]aminomethyl-1-[2-(5-(1,2,4-triazol-1-yl)-1H-indol-3-yl)ethyl]pyrrolidine;
   and pharmaceutically acceptable salts, prodrugs, and/or hydrates thereof.

The compounds disclosed herein are useful for treating and/or preventing a variety of angiogenic, microvascular and macular disorders including primary indications for diabetic retinopathy, macular degeneration (such as wet or neovascular age-related macular degeneration (AMD) and dry or atrophic AMD), macular edema, and secondary indications for inhibiting tumor vascularization, and corneal and iris neovascularization.

The most preferred compound for this invention can inhibit the binding of CXCR4 to its ligand, SDF-1, with an IC₅₀ of 7-20 nM. Ligand binding studies can be performed on crude cell membrane fractions isolated from CHO cells stably expressing human CXCR4. 50,000 cells per assay in sample buffer (PBS, 5 mM EDTA, 0.25% BSA) are mixed with 50,000 cpm of ¹²⁵I-SDF-1 protein in the presence of serial diluted compound to determine the IC₅₀. The mixture is then shaken for 60 minutes at room temperature. Following the incubation, the assay is filtered using a Packard Filtermate onto a GF/C filter plate. Once dry, scintillation fluid is added and the plate is counted in a Packard TopCounter. The IC₅₀ is determined by fitting the data to a standard competition binding curve (4-parameter fit).

Neovascular diseases of the eye, such as neovascular AMD and diabetic retinopathy, occur when the normally quiescent vessels in the retina or choroid are stimulated to proliferate within or beneath the retina. These newly formed vessels may also cause hemorrhages at the sites of neovascularization. Together, the vessel overgrowth and hemorrhaging lead to disruption of the retinal structure and vision loss.

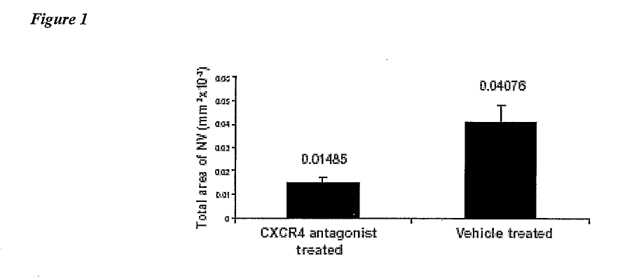

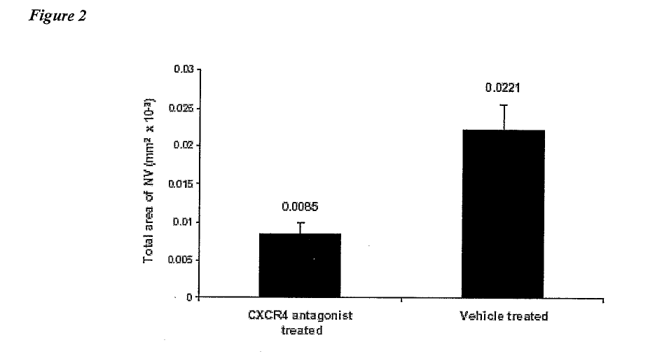

The compounds for this invention inhibit angiogenesis in an established animal model of ocular neovascularization. This model has been described previously by Kyoichi Takahashi et al., Investigative Opthalmology and Visual Science, 2003, 44: 406. Briefly, C57BL/6 mice were treated with an ophthalmic laser to produce several small breaks in Bruch's membrane to induce choroidal neovascularization. Following laser treatment, the mice received intraocular or subconjunctival injections of CXCR4 inhibitors or dosing vehicle. After 14 days, the animals were perfused with fluorescein-dextran to allow visualization of the vasculature, and their eyes were dissected and examined by fluorescence microscopy. Image analysis software was used to calculate the area of choroidal neovascularization within each lesion. FIG. 1 shows that the neovascular lesions induced by the laser procedure were significantly smaller in animals treated with subconjunctival injections of the CXCR4 inhibitor than lesions in vehicle treated animals. In this study, nine animals received 5 ul of a 1.25 mM solution of a CXCR4 inhibitor via an injection into the subconjunctival space and seventeen animals received subconjunctival injections of vehicle alone. Subconjunctival injections for the two dosing groups were performed daily for an additional 13 days. Fourteen days after the initial laser treatment, the animals were perfused with fluorescein-dextran to allow visualization of the vasculature. A Student's two-tailed t-test showed that the reduced lesion size in the CXCR4 antagonist treated mice was statistically significant as compared to the vehicle treated control animals (p=0.01). FIG. 2 shows that direct injection of a CXCR4 inhibitor into the eye (intravitreal injection) also resulted in a statistically significant decrease in neovascular lesion size as compared to vehicle treated controls (p=0.002 using the Student's two-tailed t-test). In these experiments, 1 ul of a 1.25 mM solution of a CXCR4 inhibitor was injected into the vitreous of 10 animals immediately following laser rupture of Bruch's membrane and then again on experimental day 8. Ten additional animals served as controls and had 1 ul of vehicle (phosphate buffered saline) injected intravitreally immediately following laser treatment and then again on experimental day 8. Fourteen days after the initial laser treatment, the animals were perfused with fluorescein-dextran to allow visualization of the vasculature. Taken together, these studies demonstrate that CXCR4 antagonists can inhibit ocular neovascularization.

Macular edema is a swelling of the retina that occurs within the critically important central visual zone at the posterior pole of the eye (the macula). The capillaries within the retina are composed of endothelial cells and pericytes interconnected by tight junctions. These endothelial cell:pericyte connections contribute to the blood-retinal barrier. Newly formed vessels that contain endothelial cells but that have not yet acquired a pericyte coating are more permeable and can allow the leakage of fluid and proteins which can lead to macular edema. The anti-angiogenic activities of CXCR4 inhibitors will inhibit formation of these immature, leaky vessels and potentially reduce the risk of macular edema.

The compounds produced in the present invention are readily combined with suitable and known pharmaceutically acceptable excipients to produce compositions which may be administered to mammals, including humans, to treat or prevent macular disorders. The compounds may also be combined with other angiogenesis inhibitors including, but not limited to, KDR kinase inhibitors (U.S. Pat. No. 6,306,874, incorporated herein by reference in its entirety) or angiogenic steroids such as dexamethasone, anecortave acetate, fluocinolone and triamcinolone.

Use of the compounds of formula I for the manufacture of a medicament for treating, macular edema, macular degeneration, diabetic retinopathy, corneal and iris neovascularization or for a combination thereof is also included in this invention.

Suitable subjects for the administration of the formulation of the present invention include primates, man and other animals, particularly man and domesticated animals such as cats, rabbits and dogs.

The compounds used in the instant invention can be administered in a therapeutically effective amount intravenously, subcutaneously, topically, transdermally, parenterally, or by intravitreal injection, sub-Tenon's capsule injection, periocular, retrobulbar, juxtascleral injection or any other method known to those skilled in the art. Ophthalmic pharmaceutical compositions may be adapted for localized administration to the eye in the form of solutions, suspensions, ointments, creams or as a solid or semi-solid insert. Ophthalmic formulations of this compound may contain from 0.0001 to 10% of medicament. Higher dosages as, for example, up to about 20% or lower dosages can be employed provided the dose is effective in reducing neovascularization, edema or atrophic AMD.

The pharmaceutical preparation which contains the compound may be conveniently admixed with a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, peanut oil, polyalkylene glycols, petroleum based jelly, ethyl cellulose, ethyl oleate, carboxymethyl-cellulose, polyvinylpyrrolidone, isopropyl myristate, n-methylpyrrolidone, and other conventionally employed acceptable carriers. The pharmaceutical preparation may also contain non-toxic auxiliary substances such as emulsifying, preserving, wetting, bodying agents and the like, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like. The pharmaceutical preparation may also be in the form of a microparticle or nanoparticle formulation. The pharmaceutical preparation may also be in the form of a solid or semi-solid insert. For example, one may use a solid water soluble or water insoluble polymer as the carrier for the medicament. The polymer used to form the insert may be any water soluble or water insoluble non-toxic polymer, for example, cellulose derivatives such as methylcellulose, sodium carboxymethyl cellulose, (hydroxyloweralkyl cellulose), hydroxyethyl cellulose, hydroxypropyl cellulose, hydroxypropylmethyl cellulose; acrylates such as polyacrylic acid salts, ethylacrylates, polyactylamides; natural products such as gelatin, alginates, pectins, tragacanth, karaya, chondrus, agar, acacia; the starch derivatives such as starch acetate, hydroxymethyl starch ethers, hydroxypropyl starch, as well as other synthetic derivatives such as polyvinyl alcohol, polyvinyl pyrrolidone, polyvinyl methyl ether, poly(lactide-co-glycolide), polyethylene oxide, neutralized carbopol and xanthan gum, gellan gum, and mixtures of said polymer. The pharmaceutical preparation may also be specifically designed to allow slow, sustained release from a solution, suspension or solid insert over the period of 1 day to 24 months. The pharmaceutical preparation may also be delivered via a device implanted in or near the eye such as a slow release pump, a non-biodegradable device coated with the pharmaceutical preparation, or a biodegradable or non-biodegradable device designed to control the release rate of the pharmaceutical preparation.

The pharmaceutical preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium borate, sodium acetate, sodium citrate, or gluconate buffers; and other conventional ingredients such as sodium chloride, sorbitan monolaurate, triethanolamine, polyoxyethylene sorbitan monopalmitylate, ethylenediamine tetraacetic acid, and the like.

The ophthalmic solution or suspension may be administered as often as necessary to maintain sufficient anti-neovascular, anti-vascular leakage or anti-inflammatory activity in the eye. It is contemplated that administration to the mammalian eye will be from three times daily to once every 24 months.

For topical ocular administration, the novel formulations of this invention may take the form of solutions, gels, ointments, suspensions and solid or semi-solid inserts, formulated so that a unit dosage comprises a therapeutically effective amount of the active component or some multiple thereof in the case of a combination therapy.

The formulation may also include a gum such as gellan gum at a concentration of 0.1% to 2% by weight so that the aqueous eyedrops gel on contact with the eye, thus providing the advantages of a solid ophthalmic insert as described in U.S. Pat. No. 4,861,760.

The formulation may also include a gum such as xanthan gum at a concentration of 0.1 to 2%, preferably 0.4 to 0.7% (w/w). Particularly preferred is KELTROL T xanthan gum from Monsanto Performance Materials. The formulation of the instant invention employing xanthan gum will be a hypotonic solution, with a freezing point depression between about −0.28° C. and −0.4° C., and preferably between about −0.31° C. and −0.37° C. Alternatively, the hypotonicity of the ophthalmic solutions of the present invention employing xanthan gum will be between about 150 and 215 mOs/kg, and preferably between 170 and 200 mOs/kg. Conventional ophthalmic solutions are usually prepared as isotonic solutions using tonicity adjusting agents as potassium chloride, sodium chloride, mannitol, dextrose and glycerin. An isotonic solution will have a freezing point depression of approximately −0.54 C. Tonicity may also be measured by the osmolality of the solution, an isotonic solution having an osmolality of about 290 milliosmoles per kilogram (mOs/kg).

The pharmaceutical preparation may also be in the form of a solid insert such as one which after dispensing the drug remains essentially intact as described in U.S. Pat. Nos. 4,256,108; 4,160,452; and 4,265,874; or a bio-erodible insert that either is soluble in lacrimal or vitreal fluids, or otherwise disintegrates as described in U.S. Pat. No. 4,287,175 or EPO publication 0,077,261.

The compounds used in this invention can be made in accordance with U.S. Pat. No. 5,854,268, issued Dec. 29, 1998 and herein incorporated by reference in its entirety. By way of illustration the following examples show how to prepare some of the compounds of this invention.

### EXAMPLE 1

### (3S)-3-[N—(R)-α-(Hydroxymethyl)benzyl]aminomethyl-1-[2-(5-(1,24-triazol-4-yl)-1H-indol-3-yl)ethyl]pyrrolidine

### 2.4 Hydrogen Oxalate. 0.1 Hydrate.

### 1. (3S)—N(H)-3-[(R)-α-(Hydroxymethyl)benzyl]aminomethylpyrrolidine

### a) (3S)—N-tert-Butyloxycarbonyl-3-(R)-α(hydroxymethyl)benzyl]aminomethylpyrrolidine

A solution of (R)-(−)-phenylglycinol (2.20 g, 16.1 mmol) and (3R)—N-tert-butyloxycarbonyl-3-methylsulphonyloxymethylpyrrolidine (1.0 g, 3.58 mmol), in toluene (20 ml), was heated at 150° C. for 6 h in sealed pressure tube (Aldrich). The solvent was then removed under vacuum and the residue taken up into ethyl acetate (200 ml) and washed with water (×4). The organic was dried (MgSO₄) and evaporated and the crude product chromatographed on silica gel eluting with CH₂Cl₂/MeOH (97:3) to give the title-α-(hydroxymethyl)benzylaminomethylpyrrolidine (11.0 g, 87%), δ (360 MHz, CDCl₃) 1.45 (9H, s, OC(Me)₃), 1.52-2.60 (5H, m, CH₂ and CH), 2.90-3.76 (7H, m, 3 of CH₂ and CH), 7.25-7.39 (5H, m, Ar—H).

### b) (3S)—N(H)-3-[(R)-α-Hydroxymethyl)benzyl]aminomethylpyrrolidine

Prepared from the preceding N-Boc pyrrolidine using the procedure described for Example 5, part c, δ (250 MHz, CDCl₃) 1.25-1.45 (1H, m, CH of CH₂), 1.83-1.97 (1H, m, CH of CH₂), 2.14-2.61 (4H, m, 2 of CH₂), 2.80-3.09 (3H, m, CH₂ and CH), 3.46-3.76 (3H, m, CH₂ and CH), 7.25-7.38 (5H, m, Ar—H).

### 2. (3S)-3-[N—(R)-α-(Hydroxymethyl)benzyl]aminomethyl-1-[2-(5-(1,2,4-triazol-4-yl)-1H-indol-3-yl)ethyl]pyrrolidine

### 2.4 Hydrogen Oxalate. 0.1 Hydrate

Prepared from Intermediate 3 and the preceding pyrrolidine using the procedure described for Example 41, mp 158° C., (Found: C, 55.11; H, 5.58; N, 12.85. C₂₅H₃₀N₆O.2.4(C₂H₂O₄) 0.1H₂O requires C, 55.20; H, 5.44; N, 12.96%), m/e 431 (M+1)⁺, δ (360 MHz, D₆-DMSO) 1.64-1.76 (1H, m, CH of CH₂), 2.12-2.24 (1H, m, CH of CH₂), 2.64-2.76 (2H, m, CH₂), 2.88-2.94 (1H, m, CH), 3.04-3.14 (3H, m, CH₂ and CH of CH₂), 3.30-3.42 (3H, m, CH₂ and CH of CH₂), 3.46-3.56 (1H, m, CH of CH₂), 3.73 (2H, d, J=5.7 Hz, CH₂), 4.12-4.16 (2H, m, CH₂), 7.34-7.54 (8H, m, Ar—H), 7.90 (1H, s, Ar—H), 9.04 (2H, s, Ar—H), 11.31 (1H, s, NH).

### EXAMPLE 2

### (3S)-3-[N—(S)-α-(Hydroxymethyl)benzyl]aminomethyl-1-[2-(5-(1,2,4-triazol-4-yl)-1H-indol-3-yl)ethyl]pyrrolidine

### 2.4 Hydrogen Oxalate. 0.1 Hydrate

### a) (3S)—N(H)-3-[(S)-α-(Hydroxymethyl)benzyl]aminomethylpyrrolidine

Prepared from (S)-(+)-phenylglycinol and (3R)—N-tert-butyloxycarbonyl-3-methylsulphonyloxymethylpyrrolidine using the procedures described for Example 45, part 1a.

### b) (3S)-3-[N—(S)-α-(Hydroxymethyl)benzyl]aminomethyl-1-[2-(5-(1,2,4-triazol-4-yl)-1H-indol-3-yl)ethyl]pyrrolidine

### 2.4 Hydrogen Oxalate. 0.1 Hydrate

Prepared from Intermediate 3 and the preceding pyrrolidine using the procedure described for Example 41, mp 155° C., (Found: C, 55.35; H, 5.71; N, 12.82. C₂₅H₃₀N₆O.2.4(C₂H₂O₄).0.1H₂O requires C, 55.20; H, 5.44; N, 12.96%), m/e 431 (M+1)⁺.

### EXAMPLE 3

### (3S)-3-[N-Benzyl-N-(2-hydroxy)ethyl]aminomethyl-1-[2-(5-(1,2,4-triazol-4-yl)-1H-indol-3-yl)ethyl]pyrrolidine

### 2.4 Hydrogen Oxalate

### a) (3S)—N(H)-3-[N-Benzyl-N-(2-hydroxy)ethyl]aminomethylpyrrolidine

Prepared from N-benzylethanolamine and (3R)—N-tert-butyloxycarbonyl-3-methylsulphonyloxymethylpyrrolidine using the procedures described for Example 5, parts b and c, δ (250 MHz, CDCl₃) 1.24-1.60 (2H, m, CH₂), 1.82-1.94 (2H, m, CH₂), 2.26-3.06 (9H, m, 4 of CH₂ and CH), 3.56-3.60 (2H, m, CH₂), 7.20-7.36 (5H, m, Ar—H).

### b) (3S)-3-[N-Benzyl-N-(2-hydroxy)ethyl]aminomethyl-1-[2-(5-(1,2,4-triazol-4-yl)-1H-indol-3-yl)ethyl]pyrrolidine

### 2.4 Hydrogen Oxalate

Prepared from Intermediate 3 and the preceding pyrrolidine using the procedure described for Example 41, mp 117° C., (Found: C, 55.93; H, 5.39; N, 12.50. C₂₆H₃₂N₆O.2.4(C₂H₂O₄) requires C, 55.99; H, 5.61; N, 12.72%), m/e 445 (M+1)⁺, δ (360 MHz, D₆-DMSO) 1.56-1.70 (1H, m, CH of CH₂), 2.04-2.16 (1H, m, CH of CH₂), 2.52-2.68 (7H, m, 3 of CH₂ and CH), 3.04-3.12 (2H, m, CH₂), 3.28-3.52 (6H, m, 3 of CH₂), 3.68 (2H, ABq, J=14 Hz, CH₂), 7.20-7.34 (5H, m, Ar—H), 7.38 (1H, dd, J=8.6 and 1.5 Hz, Ar—H), 7.53 (1H, d, J=8.6 Hz, Ar—H), 7.89 (1H, d, J=1.5 Hz, Ar—H), 9.03 (2H, s, Ar—H), 11.31 (1H, s, NH).

### EXAMPLE 4

### (3S)-3-(N-Phenethyl)aminomethyl-1-[2-(5-(1,2,4-triazol-4-yl)-1H-indol-3-yl)ethyl]pyrrolidine

### 2.5 Hydrogen Oxalate. Hemihydrate

### a) (3S)—N—(H)-3-(N-Phenethyl)aminomethylpyrrolidine

Prepared from phenethylamine and (3R)—N-tert-butyloxycarbonyl-3-methylsulphonyloxymethylpyrrolidine using the procedures described for Example 5, parts b and c.

### b) (3S)-3-(N-Phenethyl)aminomethyl-1-[2-(5-(1,2,4-triazol-4-yl)-1H-indol-3-yl)ethyl]pyrrolidine

### 2.5 Hydrogen Oxalate. Hemihydrate

Prepared from the preceding pyrrolidine and Intermediate 3 using the procedure described for Example 41, mp 189-190° C., (Found: C, 55.59; H, 5.55; N, 12.85. C₂₅H₃₀N₆.2.5(C₂H₂O₄)—H₂O requires C, 55.55; H, 5.59; N, 12.96%), m/e 415 (M+1)⁺, δ(360 MHz, D₆-DMSO) 1.74-1.86 (1H, m, CH of CH₂), 2.14-2.26 (1H, m, CH of CH₂), 2.68-3.60 (15H, m, CH and 7 of CH₂), 7.22-7.40 (7H, m, Ar—H), 7.53 (1H, d, J=8.6 Hz, Ar—H), 7.92 (1H, d, J=1.5 Hz, Ar—H), 9.05 (2H, s, Ar—H), 11.30 (1H, s, NH).

### EXAMPLE 5

### (3S)-3-(N-Phenethyl-N-methyl)aminomethyl-1-[2-(5-(1,2,4-triazol-4-yl)-1H-indol-3-yl)ethyl]pyrrolidine

### 2.5 Hydrogen Oxalate. 0.1 Diethyl Etherate

### a) (3S)—N(H)-3-(N-Phenethyl-N-methyl)aminomethylpyrrolidine

Prepared from N-phenethyl-N-methylamine and (3R)—N-tert-butyloxycarbonyl-3-methylsulphonyloxymethylpyrrolidine using the procedures described for Example 5, parts b and c.

### b) (3S)-3-(N-Phenethyl-N-methyl)aminomethyl-1-[2-(5-(1,2,4-triazol-4-yl)-1H-indol-3-yl)ethyl]pyrrolidine

### 2.5 Hydrogen Oxalate. 0.1 Diethyl Etherate

Prepared from the preceding pyrrolidine and Intermediate 3 using the procedure described for Example 41, mp 168-170° C., (Found: C, 57.02; H, 5.71; N, 12.78. C₂₆H₃₂N₆.2.5(C₂H₂O₄).0.1 (Diethyl ether) requires C, 57.05; H, 5.79; N, 12.71%), m/e 429 (M+1)⁺.

### EXAMPLE 6

### (3S)-3-(N-α-Dimethylbenzyl)aminomethyl-1-[2-(5-(1,2,4-triazol-4-yl)-1H-indol-3-yl)ethyl]pyrrolidine

### 2.45 Hydrogen Oxalate. 0.1 Diethyl Etherate.

Prepared from Intermediate 3 and (3R)—N-tert-butyloxycarbonyl-3-methylsulphonyloxymethylpyrrolidine using the general procedures, mp 172-174° C., (Found: C, 57.15; H, 5.94; N, 13.14. C₂₆N₃₂N₆.2.45(C₂H₂O₄).0.1 (Diethyl ether) requires C, 57.26; H, 5.82; N, 12.80%), m/e 429 (M+1)⁺, δ (360 MHz, D₆-DMSO) 1.61 (6H, s, 2 of CH₃), 1.61-1.70 (1H, m, CH of CH₂), 2.10-2.21 (1H, m, CH of CH₂), 2.54-2.62 (3H, m, CH₂ and CH), 2.96-3.48 (8H, m, 4 of CH₂), 7.30-7.57 (8H, m, Ar—H), 7.84 (1H, d, J=1.8 Hz, Ar—H), 8.92 (2H, s, Ar—H), 11.12 (1H, s, NH).

### EXAMPLE 7

### (3S)-3-(N—[S]-α-Methylbenzyl)aminomethyl-1-[2-(5-(1,2,4-triazol-1-yl)-1H-indol-3-yl)ethyl]pyrrolidine

### 2.5 Hydrogen Oxalate. 0.2 Hydrate

### a) 2-[5-(1,2,4-Triazol-1-yl)-1H-indol-3-yl]ethyl alcohol

Prepared from 4-(1,2,4-triazol-1-yl)aniline (EP497512) as described for Intermediate 3, δ (250 MHz, D₆-DMSO) 2.89 (2H, t, J=7.2 Hz, CH₂), 3.64-3.74 (2H, m, CH₂), 4.67 (1H, t, J=5.3 Hz, OH), 7.29 (1H, d, J=2.3 Hz, Ar—H), 7.47 (1H, dd, J=8.7 and 1.5 Hz, Ar—H), 7.53 (1H, dd, J=8.7 and 2.3 Hz, Ar—H), 7.95 (1H, d, J=1.9 Hz, Ar—H), 8.19 (1H, s, Ar—H), 9.19 (1H, s, Ar—H), 11.10 (1H, s, NH).

### b) (3S)-3-(N—[S]-α-Methylbenzyl)aminomethyl-1-[2-(5-(1,2,4-triazol-1-yl)-1H-indol-3-yl)ethyl]pyrrolidine

### 2.5 Hydrogen Oxalate 0.2 Hydrate

Prepared from 2-[5-(1,2,4-triazol-1-yl)-1H-indol-3-yl]ethyl alcohol and (3S)—N(H)-3-(N—[S]-α-methylbenzyl)aminomethylpyrrolidine as described for Example 41, mp 203-204° C., (Found: C, 55.95; H, 5.51; N, 13.11. C₂₅H₃₀N₆.2.5(C₂H₂O₄).0.2H₂O requires C, 56.02; H, 5.55; N, 13.07%), m/e 415 (M+1)⁺, δ (360 MHz, D₆-DMSO) 1.54 (3H, d, J=6.7 Hz, CH₃), 1.60-1.74 (1H, m, CH of CH₂), 2.11-2.22 (1H, m, CH of CH₂), 2.60-3.56 (10H, m, 4 of CH₂ and 2 of CH of CH₂), 4.24-4.30 (2H, m, CH₂), 7.34-7.56 (8H, m, Ar—H), 8.03 (1H, s, Ar—H), 8.19 (1H, s, Ar—HR), 9.19 (1H, s, Ar—H), 11.28 (1H, s, NH).

### EXAMPLE 8

### (3S)-3-[N—[R]-α(Hydroxymethyl)benzyl]aminomethyl-1-[2-(5-(1,2,4-triazol-1-yl)-1H-indol-3-yl)ethyl]pyrrolidine

### 2.0 Hydrogen Oxalate. 0.3 Hydrate

Prepared from 2-[5-(1,2,4-triazol-1-yl)-1H-indol-3-yl]ethyl alcohol and (3S)—N(H)-3-[(R)-α-(hydroxymethyl)benzyl]aminomethylpyrrolidine using the procedures described for Example 41, mp 173-174° C., (Found: C, 56.57; H, 5.77; N, 13.57. C₂₅H₃₀N₆O.2.0(C₂H₂O₄).0.3H₂O requires C, 56.54; H, 5.66; N, 13.64%), m/e 431 (M+1)⁺, δ (360 MHz, D₆-DMSO) 1.62-1.76 (1H, m, CH of CH₂), 2.10-2.22 (1H, m, CH of CH₂), 2.56-2.72 (2H, m, CH and CH of CH₂), 2.80-2.90 (1H, m, CH of CH₂), 3.02-3.52 (7H, m, 3 of CH₂ and CH), 3.64-3.70 (2H, m, CH₂), 4.02-4.06 (2H, m, CH₂), 7.32-7.57 (8H, m, Ar—H), 8.03 (1H, s, Ar—H), 8.20 (1H, s, Ar—H), 9.18 (1H, s, Ar—H), 11.28 (1H, s, NH).

### EXAMPLE 9

### (3S)-3-(N-Benzyl)aminomethyl-1-[2-(5-(1,2,4-triazol-1-ylmethyl)-1H-indol-3-yl)ethyl]pyrrolidine

### 2.4 Hydrogen Oxalate

### a) 2-[5-(1,2,4-Triazol-1-ylmethyl)-1H-indol-3-yl]ethyl alcohol

Prepared from 4-(1,2,4-triazol-1-ylmethyl)aniline (EP497512) as described for Intermediate 3, 8 (250 MHz, D₄-MeOH) 2.96 (2H, t, J=7.2 Hz, CH₂), 3.80 (2H, t, J=7.2 Hz, CH₂), 5.46 (2H, s, CH₂), 7.08 (1H, dd, J=1.7 and 8.6 Hz, Ar—H), 7.11 (1H, s, Ar—H), 7.33 (1H, d, J=8.6 Hz, Ar—H), 7.58-7.59 (1H, d, J=1.7 Hz, Ar—H), 7.97 (1H, s, Ar—H), 8.44 (1H, s, Ar—H).

### b) (3S)-3-(N-Benzyl)aminomethyl-1-[2-(5-(1,2,4-triazol-1-ylmethyl)-1H-indol-3-yl)ethyl]pyrrolidine

### 2.4 Hydrogen Oxalate

Prepared from 2-[5-(1,2,4-triazol-1-ylmethyl)-1H-indol-3-yl]ethyl alcohol and (3S)—N(H)-3-N-(benzyl)aminomethylpyrrolidine as described for Example 41, mp 154-156° C., (Found: C, 56.92; H, 5.49; N, 13.40. C₂₅H₃₀N₆.2.4(C₂H₂O₄) requires C, 56.76; H, 5.56; N, 13.33%), m/e 415 (M+1)⁺, δ (360 MHz, D₆-DMSO) 1.72-1.86 (1H, m, CH of CH₂), 2.15-2.28 (1H, m, CH of CH₂), 2.70-2.84 (1H, m, CH), 3.00-3.62 (10H, m, 5 of CH₂), 4.16 (2H, s, CH₂), 5.44 (2H, s, CH₂), 7.07 (1H, d, J=8.6 Hz, Ar—H), 7.27 (1H, s, Ar—H), 7.35 (1H, d, J=8.6 Hz, Ar—H), 7.40-7.54 (5H, m, Ar—H), 7.63 (1H, s, Ar—H), 7.95 (1H, s, Ar—H), 8.64 (1H, s, Ar—H), 11.07 (1H, s, NH).

Examples 54 and 55 were prepared from 2-[5-(1,2,4-triazol-1-ylmethyl)-1H-indol-3-yl]ethyl alcohol and the appropriate pyrrolidine using the standard procedures.

### EXAMPLE 10

### (3S)-3-(N—[S]-α-Methylbenzyl)aminomethyl-1-[2-(5-(1,2,4-triazol-1-ylmethyl)-1H-indol-3-yl)ethyl]pyrrolidine

### 2.35 Hydrogen Oxalate. 0.1 Diethyl etherate

mp: 195-197° C., (Found: C, 56.99; H, 5.65; N, 13.16. C₂₆H₃₂N₆.2.35(C₂H₂O₄).0.3(H₂O).0.1(diethyl ether) requires C, 57.21; H, 5.91; N, 12.87%), m/e 429 (M+1)⁺.

### EXAMPLE 11

### (3S)-3-(N—[R]-α-(Hydroxymethyl)benzyl]aminomethyl-1-[2-(5-(1,2,4-triazol-1-ylmethyl)-1H-indol-3-yl)ethyl]pyrrolidine

### 2.25 Hydrogen Oxalate

mp: 102-105° C., (Found: C, 56.60; H, 5.79; N, 13.02. C₂₆H₃₂N₆O.2.25(C₂H₂O₄) requires C, 56.61; H, 5.69; N, 12.99%), m/e 445 (M+1)⁺.

### EXAMPLE 12

### (3S)-3-(N-Benzyl-N-methyl)aminomethyl-1-[2-(5-(imidazol-1-yl)-1H-indol-3-yl)ethyl]pyrrolidine

### 2.0 Hydrogen Oxalate. Hemihydrate

### a) 2-[5-(Imidazol-1-yl)-1H-indol-3-yl]ethyl alcohol

Prepared from 4-(imidazol-1-yl)aniline (EP497512) as described for Intermediate 3, δ (360 MHz, D₆-DMSO) 2.87 (2H, t, J=7.2 Hz, CH₂), 3.64-3.70 (1H, m, CH₂—OH), 4.61 (1H, t, J=5.3 Hz, OH), 7.08 (1H, s, Ar—H), 7.25-7.27 (2H, m, Ar—H), 7.44 (1H, d, J=8.8 Hz, Ar—H), 7.64 (1H, d, J=2.5 Hz, Ar—H), 7.70 (1H, d, J=2.1 Hz, Ar—H), 8.11 (1H, s, Ar—H), 11.00 (1H, s, NH), m/e 228 (M+1)⁺.

### b) (3S)-3-(N-Benzyl-N-methylaminomethyl-1-[2-(5-(imidazol-1-yl)-1H-indol-3-yl)ethyl]pyrrolidine

### 2.0 Hydrogen Oxalate. Hemihydrate

To a solution of (3S)—N(H)-3-(N-methyl-N-benzyl)aminomethylpyrrolidine (0.21 g, 1.02 mmol) in anhydrous DMF (3 ml) was added K₂CO₃ (0.114 g, 0.83 mmol) and, dropwise, a solution of the mesylate of the preceding alcohol (0.168 g, 0.55 mmol) in DMF (7 ml). The mixture was heated at 50° C. for 1 h and then at 70° C. for 2 h. After cooling, the solvent was removed under vacuum and the residue partitioned between CH₂Cl₂ (3×25 ml) and water (25 ml). The combined organics were dried (Na₂SO₄) and evaporated and the residue chromatographed on silica gel eluting with CH₂Cl₂/MeOH/NH₃ (90:10:1) to give the desired product (0.134 g, 59% from the alcohol). The 2.0 hydrogen oxalate hemihydrate salt was prepared, mp 92° C. (dec.), (Found: C, 59.53; H, 6.12; N, 11.83. C₂₆H₃₁N₅.2(C₂H₂O₄).0.5H₂O requires C, 59.79; H, 6.02; N, 11.62%), m/e 414 (M+1)⁺, δ (360 MHz, D₆-DMSO) 1.60-1.74 (1H, m, CH of CH₂), 2.09-2.20 (1H, m, CH of CH₂), 2.24 (3H, s, CH₃), 2.54-3.58 (11H, m, 5 of CH₂ and CH), 3.66 (2H, ABq, J=13.3 Hz, CH₂), 7.16 (1H, s, Ar—H), 7.26-7.39 (7H, m, Ar—H), 7.51 (1H, d, J=8.5 Hz, Ar—H), 7.73 (1H, d, J=1.2 Hz, Ar—H), 7.85 (1H, d, J=2.0 Hz, Ar—H), 8.26 (1H, s, Ar—H), 11.24 (1H, s, NH).

### EXAMPLE 13

### (3S)-3-(N-Benzyl-N-methyl)aminomethyl-1-[2-(5-(1,2,4-triazol-1-ylmethyl)-1H-indol-3-yl)ethyl]pyrrolidine

### 2.5 Hydrogen Oxalate

Prepared from 2-[5-(1,2,4-triazol-1-ylmethyl)-1H-indol-3-yl]ethyl alcohol and (3S)—N(H)-3-(N-methyl-N-benzyl)aminomethylpyrrolidine using the procedure described for Example 41. The 2.0 hydrogen oxalate hemihydrate salt was prepared, mp 154-155° C., (Found: C, 57.10; H, 5.95; N, 12.66. C₂₆H₃₂N₆.2.5(C₂H₂O₄) requires C, 56.96; H, 5.70; N, 12.85%), m/e 429 (M+1)⁺, δ (360 MHz, D₆-DMSO) 1.60-1.72 (1H, m, CH of CH₂), 2.08-2.20 (1H, m, CH of CH₂), 2.26 (3H, s, CH₃), 2.52-3.60 (11H, m, 5 of CH₂ and CH), 3.69 (2H, ABq, J=13.4 Hz, CH₂), 5.42 (2H, s, CH₂), 7.05 (1H, d, J=8.5 Hz, Ar—H), 7.25-7.35 (7H, m Ar—H), 7.60 (1H, s, Ar—H), 7.92 (1H, s, Ar—H), 8.58 (1H, s, Ar—H), 11.02 (1H, s NH).

### EXAMPLE 14

### (3R)-3-(N-Methyl-N—[S]-α-methylbenzyl)aminomethyl-1-[2-(5-(1,2,4-triazol-1-ylmethyl)-1H-indol-3-yl)ethyl]pyrrolidine

### 2.0 Hydrogen Oxalate. 0.17 Diethyl etherate

### a) (3R)—N(H)-3-(N-Methyl-N—[S]-α-methylbenzyl)aminomethylpyrrolidine

Glacial acetic acid (0.9 ml, 15.7 mmol) and sodium cyanoborohydride (0.495 g, 7.88 mmol) were added successively to a stirred solution of (3S)—N-tert-butyloxycarbonyl-3-(N—[S]-α-methylbenzyl)aminomethylpyrrolidine (1.92 g, 6.31 mmol) in methanol (150 ml), at 0° C. A solution of formaldehyde (0.623 g of a 38% w/v solution, 7.88 mmol), in methanol (50 ml), was added dropwise over 0.1 h. The mixture was stirred at 0° C. for 4.5 h and then at +25° C. for 1.25 h before adding saturated K₂CO₃ solution (25 ml) and removing the solvent under vacuum. Ethyl acetate (100 ml) was added to the residue and washed with water (×1), saturated K₂CO₃ solution (×1) and brine (×1), dried (MgSO₄) and evaporated. The residue was chromatographed on silica gel eluting with CH₂Cl₂/MeOH (95:5) to give (3R)—N-tert-butyloxycarbonyl-3-(N—[S]-α-methylbenzyl-N-methyl)aminomethylpyrrolidine (2.02 g, 100%).

A solution of the preceding carbamate (2.01 g, 6.32 mmol) in 90% HCO₂H (40 ml) was stirred at 0° C. for 2.75 h and then at +25° C. for 16 h. The reaction was quenched by the addition of methanol and the solvents removed under vacuum. The residue was azeotroped with ethanol and then taken up into a small volume of water and basified with saturated K₂CO₃ solution. The aqueous was extracted with n-butanol (2×50 ml), the combined extracts evaporated in vacuo and the inorganics removed by trituration with CH₂Cl₂ and filtering. The filtrate was dried (MgSO₄) and evaporated and the residue chromatographed on silica gel, eluting with CH₂Cl₂/MeOH/NH₃ (15:8:1) to give the title pyrrolidine (1.25 g, 89%), δ (250 MHz, CDCl₃) 1.34 (3H, d, J=6.8 Hz, CH₃), 1.52-1.67 (1H, m, CH of CH₂), 1.96-2.10 (1H, m, CH of CH₂), 2.17 (3H, s, CH₃), 2.25-2.52 (3H, m, CH of CH₂), 2.72 (1H, dd, J=11.3 and 7.3 Hz, CH of CH₂), 3.10 (2H, dd, J=8.0 and 6.6 Hz, CH of CH₂), 3.25 (1H, dd, J=11.3 and 7.3 Hz, CH of CH₂), 3.57 (1H, q, J=6.8 Hz, CH), 5.97 (1H, br s, NH), 7.20-7.34 (5H, m, Ar—H).

### b) (3R)-3-(N-Methyl-N—[S]-α-methylbenzyl)aminomethyl-1-[2-5-(1,2,4-triazol-1-ylmethyl)-1H-indol-3-yl)ethyl]pyrrolidine

### 2.0 Hydrogen Oxalate. 0.17 Diethyl etherate

The title compound was prepared from the preceding pyrrolidine and the mesylate of 2-[5-(1,2,4-triazol-1-ylmethyl)-1H-indol-3-yl]ethyl alcohol using the standard coupling procedure. The 2.0 hydrogen oxalate 0.17 diethyl etherate salt was prepared, mp 148-149° C., (Found: C, 59.82; H, 6.58; N, 13.32. C₂₇H₃₄N₆.2.0(C₂H₂O₄).0.17(diethyl ether) requires C, 59.90; H, 6.30; N, 13.23%), m/e 443 (M+1)⁺, δ (360 MHz, D₆-DMSO) 1.34 (3H, d, J=6.9 Hz, CH₃), 1.60-1.71 (1H, m, CH of CH₂), 2.06-2.16 (1H, m, CH of CH₂), 2.17 (3H, s, CH₃), 2.40-2.66 (3H, m, CH of CH₂), 2.92-3.09 (3H, m, CH₂ and CH of CH₂), 3.29-3.50 (5H, m, 2 of CH₂ and CH of CH₂), 3.73 (1H, q, J=6.9 Hz, CH), 5.45 (2H, s, CH₂), 7.09 (1H, d, J=8.4 Hz, Ar—H), 7.22-7.38 (7H, m, Ar—H), 7.59 (1H, s, Ar—H), 7.91 (1H, s, Ar—H), 8.51 (1H, s, Ar—H), 10.87 (1H, s, NH).

### EXAMPLE 15

### (3R)-3-(N-Methyl-N—[R]-α-hydroxymethylbenzyl)aminomethyl-1-[2-(5-(1,2,4-triazol-1-ylmethyl)-1H-indol-3-yl)ethyl]pyrrolidine

### 1.9 Hydrogen Oxalate. Hemihydrate. 0.05 Diethyl Etherate

The title compound was prepared from (3R)—N(H)-3-(N-methyl-N—[R]-α-hydroxymethylbenzyl)aminomethylpyrrolidine and the mesylate of 2-[5-(1,2,4-triazol-1-ylmethyl)-1H-indol-3-yl)ethyl alcohol using the general procedure. The 1.9 hydrogen oxalate hemihydrate 0.05 diethyl etherate salt was prepared, mp 154-155° C., (Found: C, 57.26; H, 6.26; N, 12.75. C₂₇H₃₄N₆O.1.9(C₂H₂O₄).0.5H₂O.0.05 (diethyl ether) requires C, 57.25; H, 6.09; N, 12.92%), m/e 459 (M+1)⁺, δ (360 MHz, D₆-DMSO) 1.63-1.72 (1H, m, CH of CH₂), 2.04-2.14 (1H, m, CH of CH₂), 2.19 (3H, s, CH₃), 2.51-2.68 (3H, m, CH and CH₂), 3.00-3.10 (3H, m, CH of CH₂ and CH₂), 3.30-3.50 (5H, 2 of CH₂ and CH of CH₂), 3.63-3.89 (3H, m, CH and CH₂), 5.43 (2H, s, CH₂), 7.07 (1H, d, J=8.3 Hz, Ar—H), 7.24-7.36 (7H, m, Ar—H), 7.58 (1H, s, Ar—H), 7.89 (1H, s, Ar—H), 8.50 (1H, s, Ar—H), 10.86 (1H, s, NH).

### EXAMPLE 16

### (3R)-3-(N-Methyl-N—[S]-α-methylcyclohexylmethyl)aminomethyl-1-[2-(5-(1,2,4-triazol-1-ylmethyl)-1H-indol-3-yl)ethyl]pyrrolidine

### 2.25 Hydrogen Oxalate. 0.17 Diethyl Etherate

Prepared from 2-[5-(1,2,4-triazol-1-ylmethyl)-1H-indol-3-yl)ethyl alcohol and (3R)—N(H)-3-(N-methyl-N—[S]-α-methylcyclohexylmethyl)aminomethylpyrrolidine using previously described procedures. The 2.25 hydrogen oxalate 0.17 diethyl etherate salt was prepared, mp 191-192° C., Found: C, 58.13; H, 7.40; N, 12.80. C₂₇H₄₀N₆.2.25(C₂H₂O₄).0.17 (diethyl ether) requires C, 58.22; H, 7.02; N, 12.66%), m/e 449 (M+1)⁺, δ (360 MHz, D₆-DMSO) 0.82-0.93 (2H, m, CH₂), 0.91 (3H, d, J=6.6 Hz, CH₃), 1.09-2.40 (4H, m, 2 of CH₂), 1.56-1.74 (5H, m, 2 of CH₂ and CH of CH₂), 1.88-1.96 (1H, m, CH), 2.06-2.16 (1H, m, CH of CH₂), 2.21 (3H, s, CH₃), 2.36-2.44 (1H, m, CH), 2.48-2.62 (3H, m, CH₂ and CH of CH₂), 3.00-3.10 (3H, m, CH₂ and CH of CH₂), 3.28-3.48 (5H, m, 2 of CH₂ and CH), 5.43 (2H, s, CH₂), 7.07 (1H, dd, J=1.6 and 8.4 Hz, Ar—H), 7.24 (1H, d, J=1.6 Hz, Ar—H), 7.35 (1H, d, J=8.4 Hz, Ar—H), 7.58 (1H, s, Ar—H), 7.89 (1H, s, Ar—H), 8.49 (1H, s, Ar—H), 10.85 (1H, s, NH).

What is claimed is:

1. Use of a compound of structural formula I for the manufacture of a medicament for treating ocular disorders in a patient in need of such treatment: 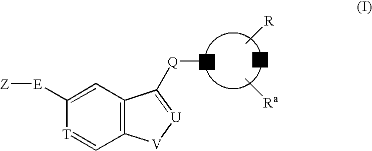
or a pharmaceutically acceptable salt, prodrug and/or hydrate thereof,
wherein
Z represents an optionally substituted five-membered heteroaromatic ring selected from furan, thiophenyl, pyrrolyl, oxazolyl, thiazolyl, isoxazolyl, isothiazolyl, imidazolyl, pyrazolyl, oxadiazolyl, thiadiazolyl, triazolyl and tetrazolyl;
E represents a chemical bond or a straight or branched alkylene chain containing from 1 to 4 carbon atoms;
Q represents a straight or branched alkylene chain containing from 1 to 4 carbon atoms, optionally substituted in any position by a hydroxy group;
T represents nitrogen or CH;
U represents nitrogen or C—R²;
V represents oxygen, sulphur or N—R³;
R² and R³ independently represent hydrogen or C₁₋₆ alkyl;
M represents the residue of an azetidine, pyrrolidine or piperidine ring;
R represents a group of formula —W—R¹;
W represents a chemical bond or a straight or branched alkylene chain containing from 1 to 4 carbon atoms, optionally substituted in any position by a hydroxy group;
R¹ represents —OR^{x}, —SR^{x}, —SOR^{x}, —SO₂R^{x} or —NR^{x}R^{y};
R^{x} and R^{y} independently represent hydrogen, hydrocarbon or a heterocyclic group; or R^{x} and R^{y} together represent a C₂₋₆ alkylene group, which alkylene group may be optionally substituted by one or more substituents selected from C₁₋₆ alkyl, aryl and hydroxy, or fused with a phenyl ring; and
R^{a} represents hydrogen, hydroxy, hydrocarbon or a heterocyclic group.

2. The use according to claim 1 wherein the ocular disorder is macular degeneration, diabetic retinopathy, or macular edema.

3. The use according to claim 2 wherein the macular degeneration is neovascular AMD or geographic atrophy, and the diabetic retinopathy is proliferative diabetic retinopathy.

4. The use according to claim 1 wherein the compound of formula I is:
(3R)-3-benzyloxy-1-[2-(5-(1,2,4-triazol-4-yl)-1H-indol-3-yl)ethyl]pyrrolidine;
(3R)-3-(4-methoxyphenyl)methoxy-1-[2-(5-(1,2,4-triazol-4-yl)-1H-indol-3-yl)ethyl]pyrrolidine;
(3R)-3-benzyloxymethyl-1-[2-(5-(1,2,4-triazol-4-yl)-1H-indol-3-yl)ethyl]pyrrolidine;
(3S)-3-(N-benzyl-N-methyl)aminomethyl-1-[2-(5-(1,2,4-triazol-4-yl)-1H-indol-3-yl)ethyl]pyrrolidine;
(2S)-2-(N-benzyl-N-methyl)aminomethyl-1-[2-(5-(1,2,4-triazol-4-yl)-1H-indol-3-yl)ethyl]pyrrolidine;
(3S)-3-(N-benzyl)aminomethyl-1-[2-(5-(1,2,4-triazol-4-yl)-1H-indol-3-yl)ethyl]pyrrolidine;
4-(4-acetylaminophenyl)methylamino-1-[3-(5-(1,2,4-triazol-4-yl)-1H-indol-3-yl)propyl]piperidine;
4-benzylamino-1-[3-(5-(1,2,4-triazol-4-yl)-1H-indol-3-yl)propyl]piperidine;
4-(N-benzyl-N-methyl)amino-1-[3-(5-(1,2,4-triazol-4-yl)-1H-indol-3-yl)propyl]piperidine;
4-(N-benzyl)aminomethyl-1-[3-(5-(1,2,4-triazol-4-yl)-1H-indol-3-yl)propyl]piperidine;
(2S)-2-(N-benzyl-N-methylaminomethyl)-1-[2-(5-(1,2,4-triazol-1-yl)-1H-pyrrolo[2,3-c]pyridin-3-yl)ethyl]pyrrolidine;
4-(N-benzyl-N-methyl)aminomethyl-1-[3-(5-(1,2,4-triazol-4-yl)-1H-indol-3-yl)propyl]piperidine;
1-[3-(5-(1,2,4-triazol-4-yl)-1H-indol-3-yl)propyl]-4-[(R)-α-(methyl)benzylamino]piperidine;
1-[3-(5-(1,2,4-triazol-4-yl)-1H-indol-3-yl)propyl]-4-[(S)-α-(methyl)benzylamino]piperidine;
1-[3-(5-(1,2,4-triazol-4-yl)-1H-indol-3-yl)propyl]-4-[(S)-α-(hydroxymethyl)benzylamino]piperidine;
1-[3-(5-(1,2,4-triazol-4-yl)-1H-indol-3-yl)propyl]-4-[(R)-α-(hydroxymethyl)benzylamino]piperidine;
1-[3-(5-(1,2,4-triazol-4-yl)-1H-indol-3-yl)propyl]-4-[(S)-(1-hydroxymethyl-2-phenyl)ethylamino]piperidine;
1-[3-(5-(1,2,4-triazol-4-yl)-1H-indol-3-yl)propyl]-4-[(1R,2S)-(2-hydroxy-1-methyl-2-phenyl)ethylamino]piperidine;
1-[3-(5-(1,2,4-triazol-4-yl)-1H-indol-3-yl)propyl]-4-[(1S,2R)-(2-hydroxy-1-methyl-2-phenyl)ethylamino]piperidine;
1-[3-(5-(1,2,4-triazol-4-yl)-1H-indol-3-yl)propyl]-4-[(1R,2R)-(2-hydroxy-1-methyl-2-phenyl)ethylamino]piperidine;
1-[3-(5-(1,2,4-triazol-4-yl)-1H-indol-3-yl)propyl]-4-[2-(4-acetylaminophenyl)ethylamino]piperidine;
1-[3-(5-(1,2,4-triazol-4-yl)-1H-indol-3-yl)propyl]-4-[(R)-α-(methyl)benzylamino]methylpiperidine;
1-[3-(5-(1,2,4-triazol-4-yl)-1H-indol-3-yl)propyl]-4-[(S)-1-(methyl)benzylamino]methylpiperidine;
1-[3-(5-(1,2,4-triazol-4-yl)-1H-indol-3-yl)propyl]-4-[(S)-1-(4-acetylaminophenyl)ethylamino]methylpiperidine;
1-[3-(5-(1,2,4-triazol-4-yl)-1H-indol-3-yl)propyl]-4-[(R)-1-(4-acetylaminophenyl)ethylamino]methylpiperidine;
1-[3-(5-(1,2,4-triazol-4-yl)-1H-indol-3-yl)propyl]-4-[N—[(R)-α-(hydroxymethyl)benzyl]-N-methylamino]piperidine;
1-[3-(5-(1,2,4-triazol-4-yl)-1H-indol-3-yl)propyl]-4-[N—[(S)-α-(hydroxymethyl)benzyl]-N-methylamino]piperidine;
1-[3-(5-(1,2,4-triazol-4-yl)-1H-indol-3-yl)propyl]-4-[N-(2-(4-acetylaminophenyl)ethyl)-N-methylamino]piperidine;
1-[3-(5-(1,2,4-triazol-4-yl)-1H-indol-3-yl)propyl]-4-[N-(4-acetylaminobenzyl)-N-methylamino]methyl piperidine;
1-[3-(5-(1,2,4-triazol-4-yl)-1H-indol-3-yl)propyl]-4-[(R)-α-(hydroxymethyl)benzylamino]methyl piperidine;
(3S)-3-(4-acetylaminobenzyl)aminomethyl-1-[2-(5-(1,2,4-triazol-4-yl)-1H-indol-3-yl)ethyl]pyrrolidine;
(3R)-3-(N-benzyl)aminomethyl-1-[2-(5-(1,2,4-triazol-4-yl)-1H-indol-3-yl)ethyl]pyrrolidine;
3-(N-benzyl)aminomethyl-1-[2-(5-(1,2,4-triazol-4-yl)-1H-indol-3-yl)ethyl]azetidine;
4-benzyl-4-hydroxy-1-[3-(5-(1,2,4-triazol-4-yl)-1H-indol-3-yl)propyl]piperidine;
3-(N-benzyl)aminomethyl-1-[3-(5-(1,2,4-triazol-4-yl)-1H-indol-3-yl)propyl]azetidine;
4-(N-benzyl)aminomethyl-4-hydroxyl-[3-(5-(1,2,4-triazol-4-yl)-1H-indol-3-yl)propyl]piperidine;
4-(N-benzyl-N-methyl)aminomethyl-4-hydroxy-1-[3-(5-(1,2,4-triazol-4-yl)-1H-indol-3-yl)propyl]piperidine;
3-(N-benzyl-N-methyl)aminomethyl-1-[2-(5-(1,2,4-triazol-4-yl)-1H-indol-3-yl)ethyl]azetidine;
(3S)-3-[N—(R)-(x-(methyl)benzyl]aminomethyl-1-[2-(5-(1,2,4-triazol-4-yl)-1H-indol-3-yl)ethyl]pyrrolidine;
(3S)-3-[N—(S)-α-(methyl)benzyl]aminomethyl-1-[2-(5-(1,2,4-triazol-4-yl)-1H-indol-3-yl)ethyl]pyrrolidine;
(3S)-3-[N—(R)-α-(hydroxymethyl)benzyl]aminomethyl-1-[2-(5-(1,2,4-triazol-4-yl)-1H-indol-3-yl)ethyl]pyrrolidine;
(3S)-3-[N—(S)-α-(hydroxymethyl)benzyl]aminomethyl-1-[2-(5-(1,2,4-triazol-4-yl)-1H-indol-3-yl)ethyl]pyrrolidine;
(3S)-3-[N-benzyl-N-(2-hydroxy)ethyl]aminomethyl-1-[2-(5-(1,2,4-triazol-4-yl)-1H-indol-3-yl)ethyl]pyrrolidine;
(3S)-3-[N-(2-phenylethyl)amino]methyl-1-[2-(5-(1,2,4-triazol-4-yl)-1H-indol-3-yl)ethyl]pyrrolidine;
(3S)-3-[N-(2-phenylethyl)-N-methylamino]methyl-1-[2-(5-(1,2,4-triazol-4-yl)-1H-indol-3-yl)ethyl]pyrrolidine;
(3S)-3-(N-x-dimethylbenzyl)aminomethyl-1-[2-(5-(1,2,4-triazol-4-yl)-1H-indol-3-yl)ethyl]pyrrolidine;
(3S)-3-[N—(S)-α-methylbenzyl]aminomethyl-1-[2-(5-(1,2,4-triazol-1-yl)-1H-indol-3-yl)ethyl]pyrrolidine;
(3S)-3-[N—(R)-α-(hydroxymethyl)benzyl]aminomethyl-1-[2-(5-(1,2,4-triazol-1-yl)-1H-indol-3-yl)ethyl]pyrrolidine;
(3S)-3-(N-benzyl)aminomethyl-1-[2-(5-(1,2,4-triazol-1-ylmethyl)-1H-indol-3-yl)ethyl]pyrrolidine;
(3S)-3-[N—(S)-α-methylbenzyl]aminomethyl-1-[2-(5-(1,2,4-triazol-1-ylmethyl)-1H-indol-3-yl)ethyl]pyrrolidine;
(3S)-3-[N—(R)-α-(hydroxymethyl)benzyl]aminomethyl-1-[2-(5-(1,2,4-triazol-1-ylmethyl)-1H-indol-3-yl)ethyl]pyrrolidine;
(3S)-3-(N-benzyl-N-methyl)aminomethyl-1-[2-(5-(imidazol-1-yl)-1H-indol-3-yl)ethyl]pyrrolidine;
(3S)-3-(N-benzyl-N-methyl)aminomethyl-1-[2-(5-(1,2,4-triazol-1-ylmethyl)-1H-indol-3-yl)ethyl]pyrrolidine;
(3R)-3-[N-methyl-N—(S)-α-methylbenzyl]aminomethyl-1-[2-(5-(1,2,4-triazol-1-ylmethyl)-1H-indol-3-yl)ethyl]pyrrolidine;
(3R)-3-[N-methyl-N—(R)-α-hydroxymethylbenzyl]aminomethyl-1-[2-(5-(1,2,4-triazol-1-ylmethyl)-1H-indol-3-yl)ethyl]pyrrolidine;
(3R)-3-[N-methyl-N—(S)-α-methylcyclohexylmethyl]aminomethyl-1-[2-(5-(1,2,4-triazol-1-ylmethyl)-1H-indol-3-yl)ethyl]pyrrolidine;
(3R)-3-[3-(R)-hydroxy-2-(R)-phenylpiperidin-1-yl]methyl-1-[2-(5-(1,2,4-triazol-1-ylmethyl)-1H-indol-3-yl)ethyl]pyrrolidine;
(3R)-3-[3-(R)-hydroxy-2-(R)-phenylpiperidin-1-yl]methyl-1-[2-(5-(1,2,4-triazol-1-yl)-1H-indol-3-yl)ethyl]pyrrolidine;
4-hydroxy-4-(phenylsulfinyl)methyl-1-[3-(5-(1,2,4-triazol-4-yl)-1H-indol-3-yl)propyl]piperidine;
(3R)-3-[2-(R,S)-phenylpiperidin-1-yl]methyl 1-[2-(5-(1,2,4-triazol-1-ylmethyl)-1H-indol-3-yl)ethyl]pyrrolidine;
4-(3,3-dimethylpiperidin-1-yl)methyl-4-hydroxy-1-[3-(5-(1,2,4-triazol-4-yl)-1H-indol-3-yl)propyl]piperidine;
4-hydroxy-4-(1,2,3,4-tetrahydroisoquinolin-2-yl)methyl-1-[3-(5-(1,2,4-triazol-4-yl)-1H-indol-3-yl)propyl]piperidine;
4-hydroxy-4-(N-isobutyl-N-methyl)aminomethyl-1-[3-(5-(1,2,4-triazol-4-yl)-1H-indol-3-yl)propyl]piperidine;
4-[N-benzyl-N-(2-hydroxyethyl)amino]methyl-4-hydroxy-1-[3-(5-(1,2,4-triazol-4-yl)-1H-indol-3-yl)propyl]piperidine;
4-[N-(2,2-dimethylpropyl)-N-methylamino]methyl-4-hydroxy-1-[3-(5-(1,2,4-triazol-4-yl)-1H-indol-3-yl)propyl]piperidine;
4-[N—(R)-α-hydroxymethylbenzyl-N-methylamino]methyl-4-hydroxy-1-[3-(5-(1,2,4-triazol-4-yl)-1H-indol-3-yl)propyl]piperidine;
4-hydroxy-4-(2-methylphenylmethyl)aminomethyl-1-[3-(5-(1,2,4-triazol-4-yl)-1H-indol-3-yl)propyl]piperidine;
4-hydroxy-4-[N-(2-methylphenylmethyl)-N-methylamino]methyl-1-[3-(5-(1,2,4-triazol-4-yl)-1H-indol-3-yl)propyl]piperidine;
3-(benzylamino)methyl-3-hydroxy-1-[3-(5-(1,2,4-triazol-4-yl)-1H-indol-3-yl)propyl]pyrrolidine;
3-(benzylamino)methyl-3-hydroxy-1-[2-(5-(1,2,4-triazol-4-yl)-1H-indol-3-yl)ethyl]pyrrolidine;
1-[3-(5-(1,2,4-triazol-4-yl)-1H-indol-3-yl)propyl]-4-[(R)-α-(carbamoyl-oxymethyl)benzylamino]piperidine;
1-[3-(5-(1,2,4-triazol-4-yl)-1H-indol-3-yl)propyl]-4-[(1R,2S)-2-hydroxy-1-phenylpropylamino]piperidine;
1-[3-(5-(1,2,4-triazol-4-yl)-1H-indol-3-yl)propyl]-4-[(1R,2R)-2-hydroxy-1-phenylpropylamino]piperidine;
1-[3-(5-(1,2,4-triazol-4-yl)-1H-indol-3-yl)propyl]-4-[(R,S)-1-hydroxy-2-phenylprop-2-ylamino]piperidine;
1-[3-(5-(1,2,4-triazol-4-yl)-1H-indol-3-yl)propyl]-4-[(R)-2-hydroxy-1-(4-fluorophenyl)ethylamino]piperidine;
1-[3-(5-(1,2,4-triazol-4-yl)-1H-indol-3-yl)propyl]-4-[(1R,2R)-2-hydroxyindan-1-ylamino)piperidine;
1-[3-(5-(1,2,4-triazol-4-yl)-1H-indol-3-yl)propyl]-4-[(R,S)-indan-1-ylamino]piperidine;
1-[3-(5-(1,2,4-triazol-4-yl)-1H-indol-3-yl)propyl]-4-[(R,S)-1-(4-fluorophenyl)ethylamino]piperidine;
1-[3-(5-(1,2,4-triazol-4-yl)-1H-indol-3-yl)propyl]-4-[(R)-1-phenylprop-2-ylamino]piperidine;
1-[3-(5-(1,2,4-triazol-4-yl)-1H-indol-3-yl)propyl]-4-[N-(3,3-dimethylallyl)-N-methyl o]piperidine;
1-[3-(5-(1,2,4-triazol-4-yl)-1H-indol-3-yl)propyl]-4-(N-allyl-N-methylamino)piperidine;
1-[3-(5-(1,2,4-triazol-4-yl)-1H-indol-3-yl)propyl]-4-(indan-1-ylaminomethyl)piperidine;
1-[3-(5-(1,2,4-triazol-4-yl)-1H-indol-3-yl)propyl]-4-[N—(R)-α-(hydroxymethyl)benzyl-N-methylaminomethyl]piperidine;
(3R)-3-(benzylthio)methyl-1-[2-(5-(1,2,4-triazol-4-yl)-1H-indol-3-yl)ethyl]pyrrolidine;
(±)-1-[3-(5-(1,2,4-triazol-4-yl)-1H-indol-3-yl)propyl]-4-(1-benzylamino-2-hydroxyethyl)piperidine;
1-[3-(5-(1,2,4-triazol-1-yl)-1H-indol-3-yl)propyl]-4-[(R)-α-(hydroxymethyl)benzylamino]piperidine;
1-[3-(5-(imidazol-1-yl)-1H-indol-3-yl)propyl]-4-[(R)-α-(methyl)benzylamino]piperidine;
1-[3-(5-(imidazol-1-yl)-1H-indol-3-yl)propyl]-4-[(R)-α-(hydroxymethyl)benzylamino]piperidine;
1-[3-(5-(1,2,4-triazol-1-ylmethyl)-1H-indol-3-yl)propyl]-4-[(R)-α-(hydroxymethyl)benzylamino]piperidine;
1-[3-(5-(1,2,4-triazol-4-yl)-1H-indol-3-yl)propyl]-4-[(R)-α-(methoxymethyl)benzylamino]piperidine;
1-[3-(5-(1,2,4-triazol-4-yl)-1H-indol-3-yl)propyl]-4-[N—(R)-α-(methoxymethyl)benzyl-N-methylamino]piperidine;
1-[3-(5-(imidazol-1-yl)-1H-indol-3-yl)propyl]-4-[(R)-α-(methoxymethyl)benzylamino]piperidine;
1-[3-(5-(1,2,4-triazol-1-ylmethyl)-1H-indol-3-yl)propyl]-4-[(R)-1-(4-fluorophenyl)-2-methoxyethylamino]piperidine;
1-[3-(5-(1,2,4-triazol-1-ylmethyl)-1H-indol-3-yl)propyl]-4-[N-(4-fluorobenzyl)-N-methylamino]piperidine;
1-[3-(5-(1,2,4-triazol-4-yl)-1H-indol-3-yl)propyl]-4-(2-phenylpiperidin-1-yl)piperidine;
1-[3-(5-(1,2,4-triazol-4-yl)-1H-indol-3-yl)propyl]-4-[(R)-1-(4-fluorophenyl)-2-methoxyethylamino]piperidine;
(3R)-3-(benzylsulfinyl)methyl-1-[2-(5-(1,2,4-triazol-4-yl)-1H-indol-3-yl)ethyl]pyrrolidine;
(3R)-3-(4-fluorobenzylthio)methyl-1-[2-(5-(1,2,4-triazol-1-ylmethyl)-1H-indol-3-yl)ethyl]pyrrolidine;
(3R)-3-(4-fluorobenzylsulfinyl)methyl-1-[2-(5-(1,2,4-triazol-1-ylmethyl)-1H-indol-3-yl)ethyl]pyrrolidine;
(3R)-3-(4-fluorobenzylsulfonyl)methyl-1-[2-(5-(1,2,4-triazol-1-ylmethyl)-1H-indol-3-yl)ethyl]pyrrolidine;
4-(4-fluorobenzylsulfinyl)-1-[3-(5-(1,2,4-triazol-4-yl)-1H-indol-3-yl)propyl]piperidine;
and pharmaceutically acceptable salts, prodrugs, and/or hydrates thereof.

5. The use according to claim 4 wherein the compound of formula I is:
(3S)-3-[N-(2-phenylethyl)amino]methyl-1-[2-(5-(1,2,4-triazol-4-yl)-1H-indol-3-yl)ethyl]pyrrolidine;
(3S)-3-[N-(2-phenylethyl)-N-methylamino]methyl 1-[2-(5-(1,2,4-triazol-4-yl)-1H-indol-3-yl)ethyl]pyrrolidine;
(3S)-3-(N-α-dimethylbenzyl)aminomethyl-1-[2-(5-(1,2,4-triazol-4-yl)-1H-indol-3-yl)ethyl]pyrrolidine;
(3S)-3-[N—(S)-α-methylbenzyl]aminomethyl-1-[2-(5-(1,2,4-triazol-1-yl)-1H-indol-3-yl)ethyl]pyrrolidine;
(3S)-3-[N—(R)-(x-(hydroxymethyl)benzyl]aminomethyl-1-[2-(5-(1,2,4-triazol-1-yl)-1H-indol-3-yl)ethyl]pyrrolidine;
(3S)-3-(N-benzyl)aminomethyl-1-[2-(5-(1,2,4-triazol-1-ylmethyl)-1H-indol-3-yl)ethyl]pyrrolidine;
(3S)-3-[N—(S)-α-methylbenzyl]aminomethyl-1-[2-(5-(1,2,4-triazol-1-ylmethyl)-1H-indol-3-yl)ethyl]pyrrolidine;
(3S)-3-[N—(R)-α-(hydroxymethyl)benzyl]aminomethyl-1-[2-(5-(1,2,4-triazol-1-ylmethyl)-1H-indol-3-yl)ethyl]pyrrolidine;
(3S)-3-(N-benzyl-N-methyl)aminomethyl-1-[2-(5-(imidazol-1-yl)-1H-indol-3-yl)ethyl]pyrrolidine;
(3S)-3-(N-benzyl-N-methyl)aminomethyl-1-[2-(5-(1,2,4-triazol-1-ylmethyl)-1H-indol-3-yl)ethyl]pyrrolidine; and salts and prodrugs thereof.
and pharmaceutically acceptable salts, prodrugs, and/or hydrates thereof.

6. The use according to claim 5 wherein the compound is (3S)-3-(N-α-dimethylbenzyl)aminomethyl-1-[2-(5-(1,2,4-triazol-4-yl)-1H-indol-3-yl)ethyl]pyrrolidine

7. Use of a compound of claim 1 for the manufacture of a medicament for the treatment of diabetic retinopathy, corneal neovascularization or iris neovascularization in a patient in need of such treatment.
